(19) United States
(12) Patent Application Publication
(10) Pub. No.: US 2024/0342255 A1
(43) Pub. Date: Oct. 17, 2024

(54) TARGETING 3-KETODIHYDROSPHINGOSINE REDUCTASE (KDSR) IN CANCER

(71) Applicant: UNIVERSITY OF MASSACHUSETTS, Boston, MA (US)

(72) Inventors: Dohoon KIM, Shrewsbury, MA (US); Meghan SPEARS, Boston, MA (US); Namgyu LEE, Worcester, MA (US)

(21) Appl. No.: 18/682,548

(22) PCT Filed: Aug. 11, 2022
(86) PCT No.: PCT/US22/40112

§ 371 Date: Feb. 9, 2024

Related U.S. Application Data
(60) Provisional application No. 63/232,413, filed on Aug. 12, 2021.

(51) Int. Cl. A61K 38/46; A61K 31/20; A61K 31/713; A61K 45/06; A61P 35/00; C12N 15/113; C12N 15/86

(52) U.S. Cl. CPC A61K 38/465; A61K 31/20; A61K 31/713; A61K 45/06; A61P 35/00; C12N 15/1137; C12N 15/86; C12N 2740/15043

(57) ABSTRACT

Provided herein are methods for treating cancer using an inhibitor of KDSR, a precursor of 3KDS (e.g., palmitate), 3KDS itself, or a combination thereof.

### CROSS-REFERENCE TO RELATED APPLICATIONS

This application claims the benefit of U.S. Provisional Patent Application No. 63/232,413, filed Aug. 12, 2021. The contents of this application are incorporated herein by reference in its entirety.

### FIELD OF THE INVENTION

The subject matter disclosed herein generally relates to methods and compositions for treating cancer.

### BACKGROUND OF THE INVENTION

Sphingolipids are a class of lipid molecules that contain a long chain ‘sphingoid’ base backbone and have extensive and complex metabolism^{1,2}. As a class of bioactive lipids, they have established roles in many cellular processes including as signaling molecules such as ceramides and sphingosine 1-phosphate and as membrane components such as sphingomyelin^{3,4}. Many studies have demonstrated that sphingolipids can regulate various cancer-associated processes, both pro- and anti-tumorigenic, including cell proliferation, survival, migration, and invasion, as well as cell cycle arrest and cell death^{4,5}. Sphingolipids can be produced de novo through an ER localized biosynthesis pathway or obtained through salvage pathways⁶. Although it is recognized that sphingolipids have roles in cancer, because they can be salvaged from the extracellular environment, the importance specifically of their de novo biosynthesis in cancer is largely unknown.

### SUMMARY OF THE INVENTION

The present disclosure is based, at least in part, on the surprising discovery that endoplasmic reticulum (ER) function and proteostasis depend on a critical detoxification step that occurs during sphingolipid biosynthesis, which can be exploited to kill cancer cells. It was found that 3-ketodihydrosphingosine reductase (KDSR), the enzyme that processes the sphingolipid biosynthesis precursor 3-ketodihydrosphingosine (3KDS), is essential to the survival of some cancer cell types. The role of KDSR in de novo sphingolipid biosynthesis per se was dispensable to cancer cells as they readily acquired sphingolipids through salvage. Rather, KDSR is required to prevent a toxic buildup of 3KDS, which in turn results in collapse of the ER and breakdown of the ubiquitin proteasome system. This can be exploited to selectively poison cancer cells, such as breast cancer cells, which have elevated expression of the enzyme serine palmitoyltransferase (SPT: a complex of the structural subunit SPTLC1 and the catalytic subunit SPTLC2: knockout of either will completely ablate the enzyme activity) which produces 3KDS. Disruption of KDSR induces 3KDS accumulation in vivo, and dramatically impairs tumor growth, which can be synergized with a high fat diet providing additional palmitate input for 3KDS production. Thus, restricting buildup of the toxic metabolite 3KDS during sphingolipid biosynthesis is critical in maintaining ER function and proteostasis, which may be therapeutically exploited.

Accordingly, aspects of the present disclosure provide methods for treating a cancer, the method comprising administering to a subject in need thereof an effective amount of an inhibitor of 3-ketodihydrosphingosine reductase (KDSR), a precursor of 3-ketodihydrosphingosine (3KDS), 3KDS, or a combination thereof.

In some embodiments, the inhibitor of KDSR is selected from the group consisting of a small molecule inhibitor, a peptide inhibitor, an anti-KDSR antibody or antigen binding fragment thereof, and an agent that inhibits expression of KDSR.

In some embodiments, the agent that inhibits expression of KDSR is selected from the group consisting of short interfering nucleic acid (siNA), short interfering RNA (siRNA), double-stranded RNA (dsRNA), micro-RNA (miRNA), and short hairpin RNA (shRNA).

In some embodiments, the agent that inhibits expression of KDSR is a guide RNA (gRNA), and the method comprises further administering to the subject a clustered regularly interspaced short palindromic repeat (CRISPR)-associated 9) (Cas9) nuclease. In some embodiments, the Cas9 nuclease is a nucleic acid encoding the Cas9 nuclease. In some embodiments, the nucleic acid encoding the Cas9 nuclease is comprised in a vector. In some embodiments, the vector is a viral vector. In some embodiments, the viral vector is selected from the group consisting of a retroviral vector, a lentiviral vector, an adenoviral vector, an adeno-associated viral vector, a herpes simplex viral vector, and a vaccinia viral vector.

In some embodiments, the precursor of 3KDS is palmitate.

In some embodiments, the inhibitor of KDSR, the precursor of 3KDS, 3KDS, or a combination thereof is formulated in a pharmaceutical composition, which further comprises a pharmaceutically acceptable carrier.

In some embodiments, the cancer is selected from the group consisting of colorectal cancer, fibrosarcoma, lung cancer, brain cancer, breast cancer, and prostate cancer.

In some embodiments, the subject is a human patient.

In some embodiments, methods provided herein further comprise administering to the subject an additional therapy. In some embodiments, the additional therapy is selected from the group consisting of radiation therapy, surgical therapy, chemotherapy, and immunotherapy.

In some embodiments, the subject consumes a high fat diet or ketogenic diet.

### DETAILED DESCRIPTION OF THE DRAWINGS

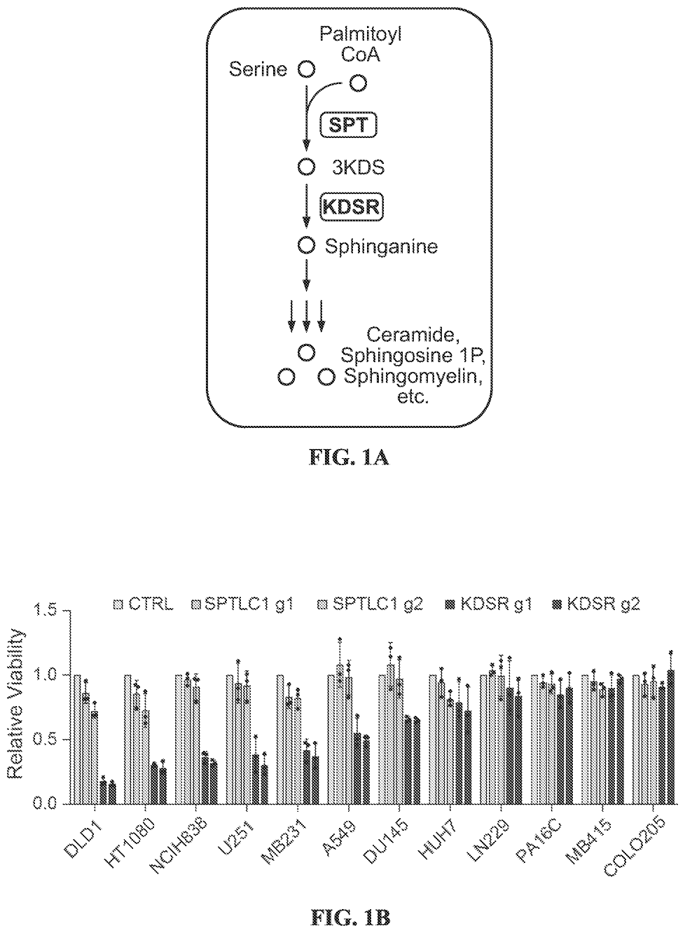

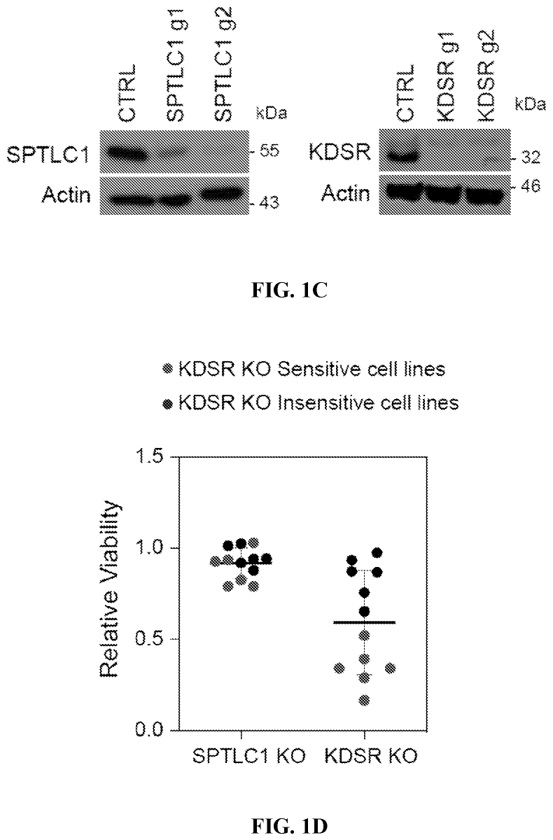

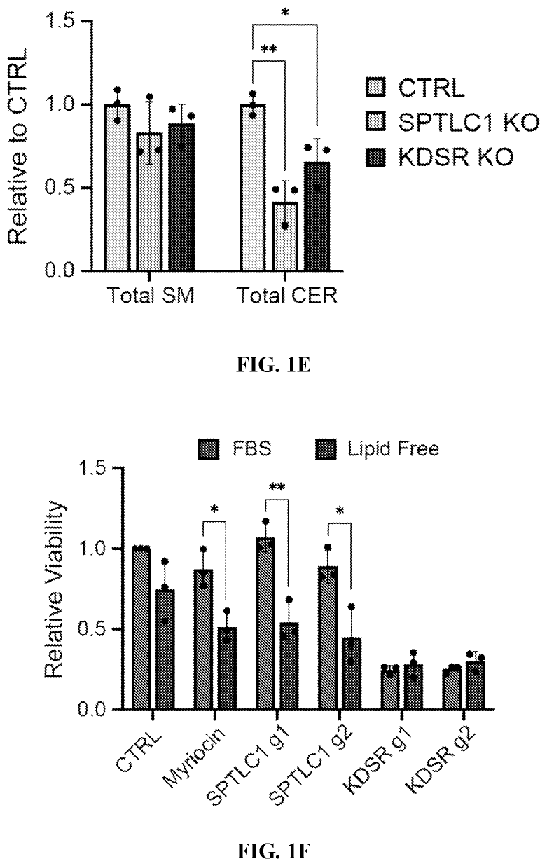

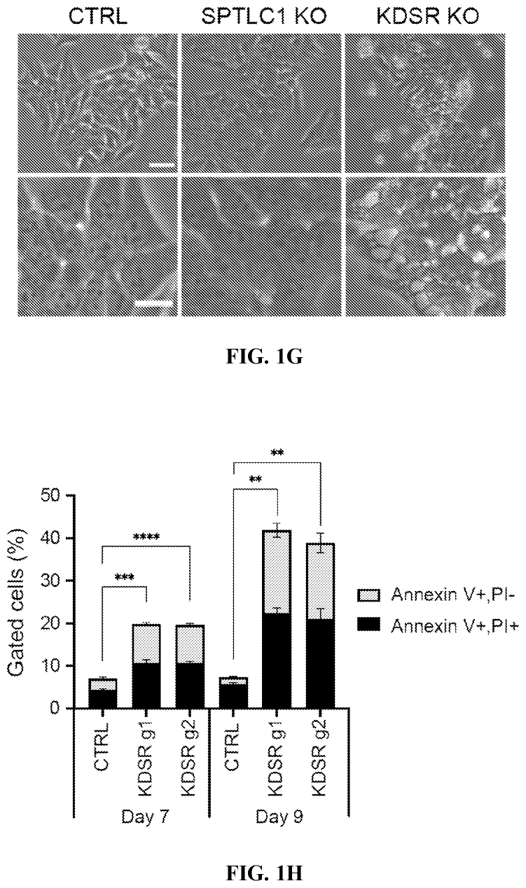

FIGS. 1A-1I: Disruption of sphingolipid biosynthesis enzyme KDSR is deleterious to a subset of cancer cells despite their capacity to salvage sphingolipids. FIG. 1A: Simplified diagram depicting the de novo sphingolipid biosynthesis pathway. Begins with serine and palmitoyl-CoA as substrates for the SPT enzyme complex to produce 3KDS, which is subsequently converted to sphinganine by the enzyme KDSR, followed by the downstream production of a variety of sphingolipid species. (Created with Biorender). FIG. 1B: Viabilities of 12 cancer cell lines of various tissue origins following CRISPR-Cas9 mediated gene KO of SPTLC1 (grey) or KDSR (black) with 2 guide RNAs (g1 and g2) relative to a non-targeting control guide (CTRL, light gray), which is set at 1.0. FIG. 1C: Western blot showing protein levels of SPTLC1 and KDSR in CTRL. SPTLC1 KO, and KDSR KO DLD1 cells with actin as the loading control. FIG. 1D: Dot plot summarizing the viabilities of 12 cancer cell lines (depicted in FIG. 1B) with SPTLC1 and KDSR KO as an average of g1 and g2 KO relative to CTRL. Each dot represents one cell line and is depicted as KDSR KO sensitive (greater than 40% loss of viability, red) or insensitive (less than 40% loss of viability, black). FIG. 1E: Quantification of total sphingomyelin (SM) and ceramide (CER) levels by LC-MS in SPTLC1 KO and KDSR KO DLD1 cells relative to CTRL (set at 1.0). FIG. 1F: Viability of DLD1 cells subjected to 15 μM myriocin treatment, CTRL, SPTLC1 KO, or KDSR KO grown in media with 10% FBS (light gray) or 10% charcoal stripped serum (lipid free, dark gray). Values are relative to the viability of CTRL cells grown in 10% FBS (set at 1.0). FIG. 1G: Light microscope images of DLD1 cells with CTRL, SPTLC1 KO, or KDSR KO, displaying the morphology of the cells at 2 magnifications. Top row scale bar=50 μm, bottom row scale bar=20 μm. FIG. 1H: Percentages of Annexin V+,PI− (light gray) and Annexin V+/PI+ (black) DLD1 cells subjected to CTRL or KDSR KO with 2 guides at day 7 and day 9 post selection with puromycin after lentiviral transduction. FIG. 1I: Relative viability of DLD1 cells overexpressing empty pLV vector or g1-resistant KDSR subjected to non-targeting CTRL gRNA or KDSR g1 CRISPR gene KO. Viabilities are shown as relative to CTRL cells overexpressing empty vector set at 1.0. For FIG. 1B, FIG. 1F, and FIG. 1I, each point represents the average of technical replicates from an independent experiment for n=3 biological replicates. For FIG. 1E and FIG. 1H, n=3 independently prepared biological replicates. Data are shown as mean±s.d. P values were calculated using two-tailed Student's t test (*p<0.05, **P<0.01, ***p<0.001, ****P<0.0001).

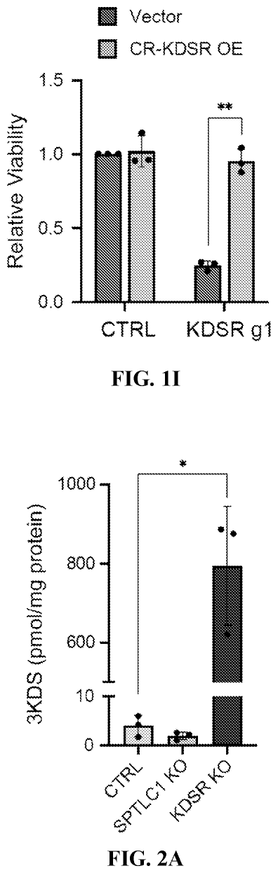

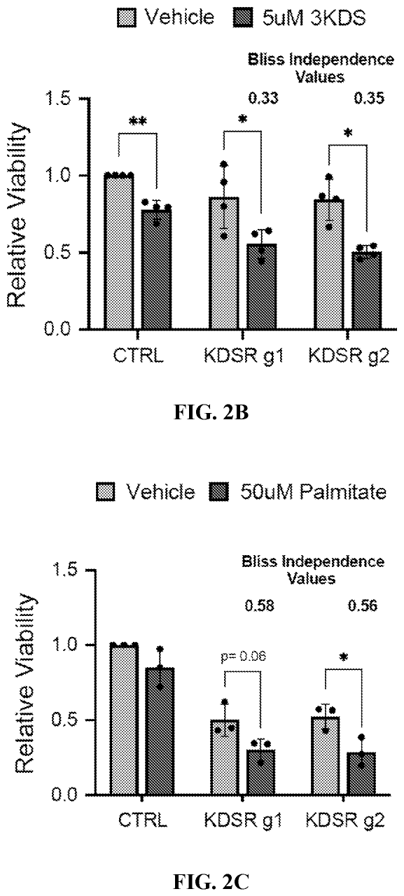

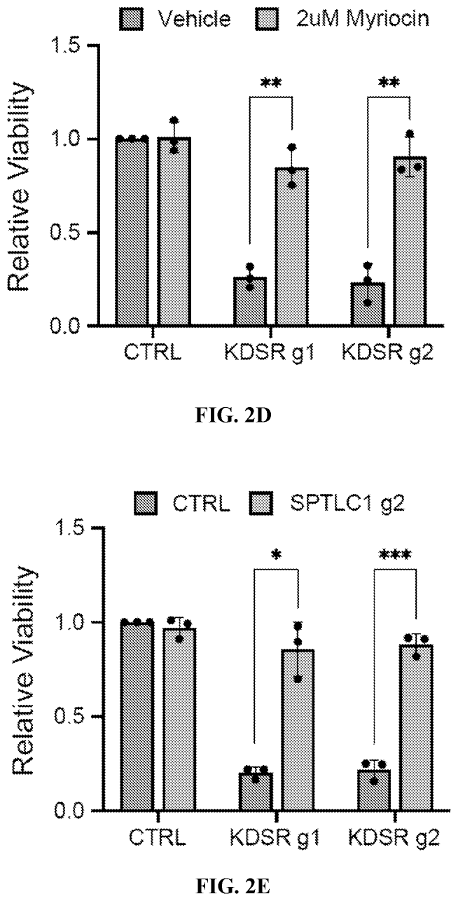

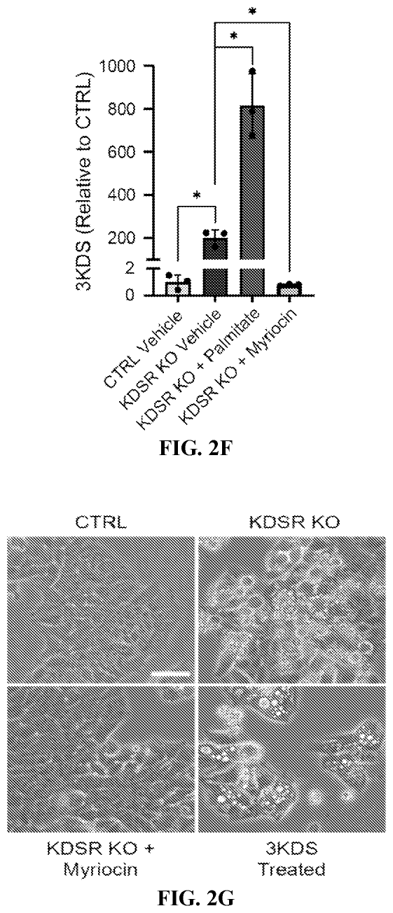

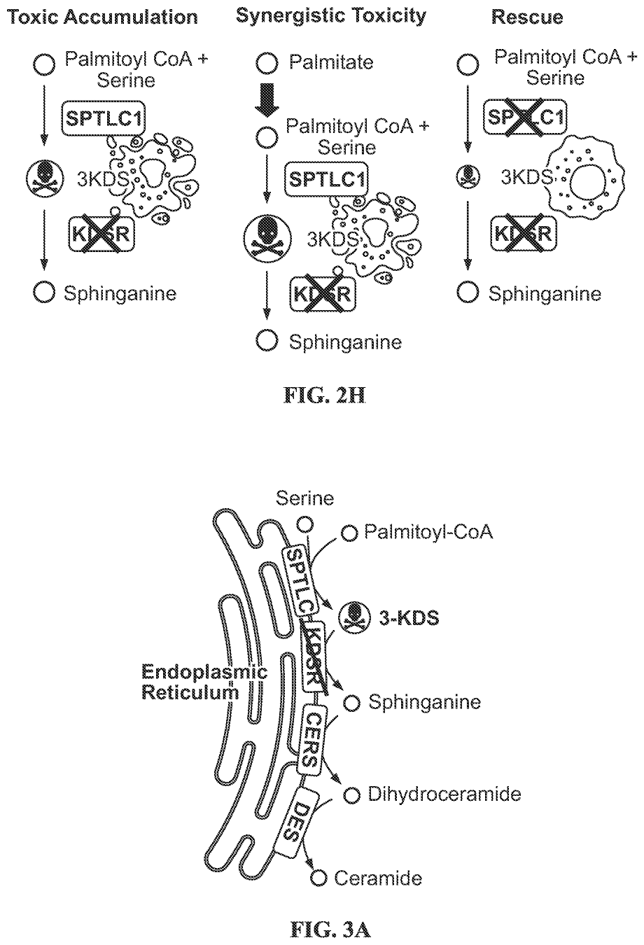

FIG. 2A-2H: KDSR is required for 3KDS detoxification rather than to prevent sphingolipid starvation. FIG. 2A: Quantification of the levels of 3KDS by LC-MS in DLD1 cells subjected to non-targeting CTRL gRNA (light gray). SPTLC1 g1 (dark gray), or KDSR g1 (black) mediated gene KO. FIG. 2B: Percent viability of DLD1 cells treated with various doses of 3KDS for 24 hr. Percent viability is relative to control untreated cells set at 100%. FIG. 2C: Relative viability of CTRL. KDSR g1, and KDSR g2 KO DLD1 cells treated with EtOH vehicle (light gray) or 50 μM palmitate (dark gray) for 48 hr. Viabilities are shown relative to CTRL DLD1 cells treated with EtOH vehicle, set at 1.0. Bliss independence values were calculated using the equation EC=EA+EB−EA×EB, where EC is the expected loss of viability if the effect of 2 conditions act independently of each other. EA is the loss of viability with KDSR KO, and EB is the loss of viability with palmitate treatment. Using this equation, synergy is defined as Eobserved>EC. FIG. 2D: Relative viability of DLD1 cells subjected to CTRL gRNA or KDSR g1 and g2 mediated gene KO treated with DMSO vehicle (dark gray) or 2 μM of the SPT inhibitor myriocin (light gray) for 72 hr. Viabilities are shown relative to CTRL DLD1 cells treated with DMSO vehicle, set at 1.0. FIG. 2E: Relative viability of DLD1 cells subjected to sequential gene KO with gRNA against CTRL or SPTLC1 g2, followed by KO with gRNA against CTRL. KDSR g1, or g2. Viabilities are shown relative to DLD1 cells with CTRL KO followed by CTRL KO set at 1.0. FIG. 2F: Quantification of relative 3KDS levels in DLD1 CTRL cells treated with vehicle (light gray). KDSR KO cells treated with vehicle (dark gray), KDSR KO cells treated with 50 μM Palmitate (dark gray), and KDSR KO cells treated with 15 μM myriocin (light gray). The values for CTRL and KDSR KO vehicle treated conditions are the same as those used in FIG. 2A, but here are shown relative to CTRL vehicle set at 1.0, in order to display additional changes induced by palmitate and myriocin treatment. FIG. 2G: Light microscope images showing the morphology of CTRL and KDSR KO DLD1 cells treated with DMSO vehicle (48 hr). KDSR KO DLD1 cells treated with 15 μM myriocin (48 hr), and wildtype DLD1 cells treated with 10 μM 3KDS (24 hr). All images show the same magnification, scale bar=50 μm. Image of 3KDS treated cells were taken at a different time in a different experiment than the other 3 conditions. FIG. 2H: Schematic of proposed toxicity models for KDSR KO, including synergy of palmitate treatment representing data in panel c, and rescue of disruption of 3KDS production through SPT inhibition, representing data in FIGS. 2D-2E. (Created with Biorender). For FIGS. 2B-2E, each point represents the average of 3 technical replicates from an independent experiment for n=2 biological replicates in b and n=3 biological replicates in FIGS. 2C-2E. For FIG. 2A and FIG. 2F, the quantification of 3KDS was normalized to the protein content of n=3 independently prepared cell extracts. Where applicable, data are shown as mean+s.d. p values were calculated using two-tailed Student's t test for all statistics shown (*p<0.05, **P<0.01, ***p<0.001).

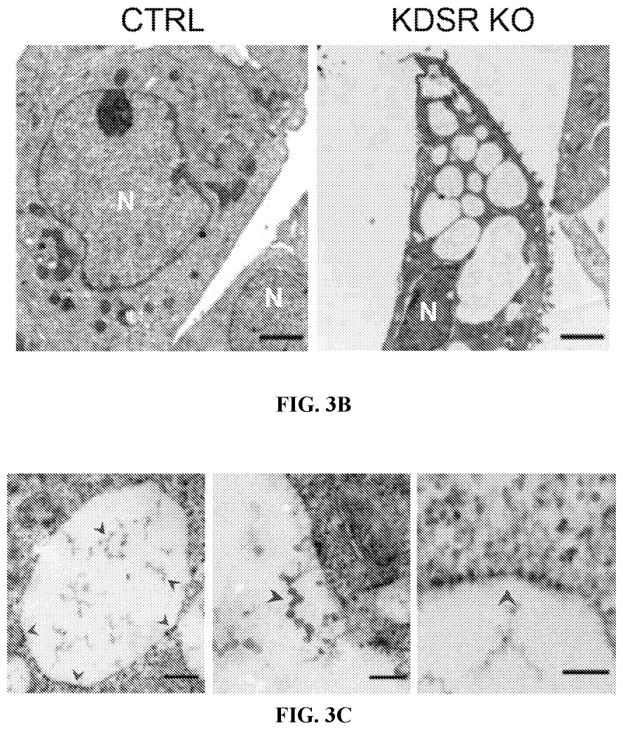

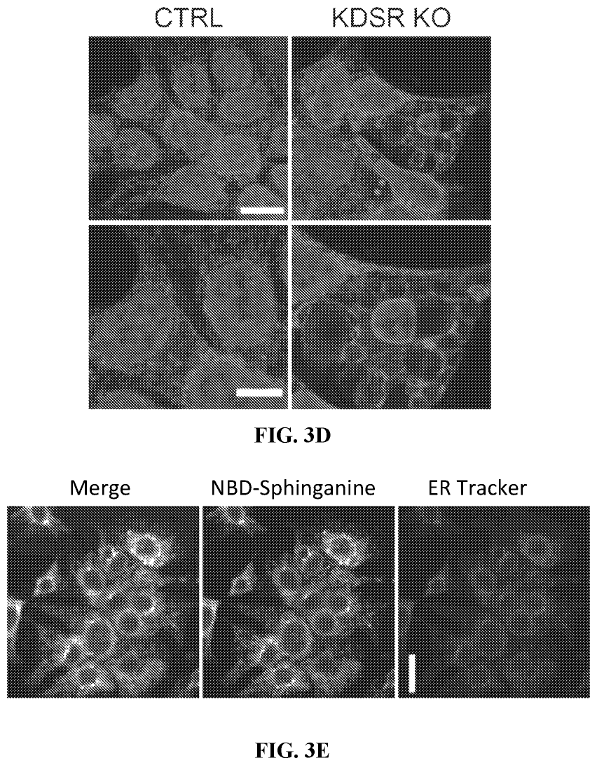

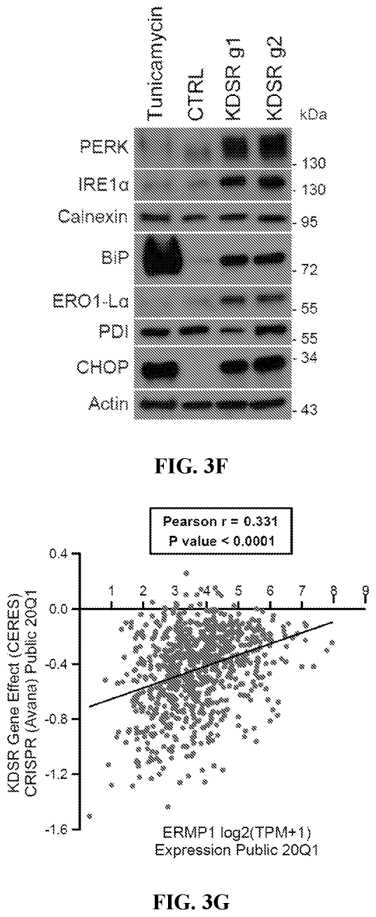

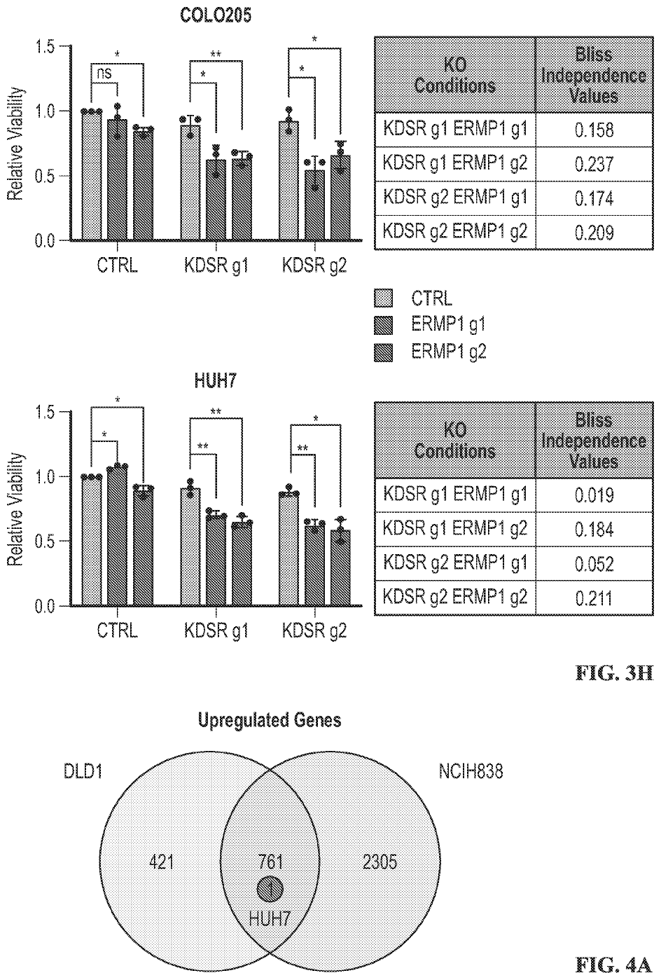

FIGS. 3A-3H: Accumulation of 3KDS severely disrupts ER structure and function. FIG. 3A: Simplified diagram depicting localization of KDSR and the de novo sphingolipid biosynthesis pathway along the cytosolic side of the ER membrane. (Created with Biorender). FIG. 3B: Representative electron microscope images of DLD1 CTRL and KDSR KO cells, depicting an overview of the aberrant morphology observed by light microscope only in cells with KDSR KO. N indicates nucleus. Scale bar=3 μm. FIG. 3C: Magnified electron microscope images displaying the features of the subcellular structures in KDSR KO DLD1 cells. Red arrows point to areas where ribosomes line the edges of the structures, blue arrows point to potential polysome chains near the structures. Left image scale bar=0.2 μm, middle image scale bar=0.2 μm, right image scale bar=0.1 μm. FIG. 3D: Representative immunofluorescent images of CTRL and KDSR KO cells immunostained for Calnexin (ER membrane) and DAPI (nucleus). Cells are shown at 2 magnifications to depict an overview and close up view of ER structure under each condition. Top row scale bar=15 μm, bottom row scale bar=8 μm. FIG. 3E: Representative immunofluorescent images of wildtype DLD1 cells that were simultaneously stained with ER tracker and treated with NBD-sphinganine. Top 2 images show ER individual channels with DAPI staining for clarity of cell positioning, and the bottom image shows the merge of all 3 channels. Scale bar=20 μm. FIG. 3F: Western blot showing protein expression of characteristic markers of ER stress in lysates from DLD1 cells subjected to CTRL and KDSR g1 and g2 mediated KO. Lysates from DLD1 cells treated with 2 μM Tunicamycin for 24 hr were also probed as a positive control for ER stress induction. These 4 samples were run under the same conditions on 2 separate blots in order to detect all 9 antibodies, with actin as the loading control. FIG. 3G: Correlation plot showing the relationship between the effect of CRISPR KO of KDSR (y axis) and the mRNA expression of its top correlate ERMP1 (x axis) in n=800 cancer cell lines (represented by gray dots). This relationship was identified using the Broad Institute's Cancer Dependency Map (depmap.org/portal/). Black line shows linear regression. Pearson r =0.331, p<0.0001. FIG. 3H: Relative viability of COLO205 and HUH7 (KDSR KO insensitive) cells subjected to sequential gene KO with gRNA against CTRL, ERMP1 g1, or g2, followed by KO with gRNA against CTRL. KDSR g1, or g2. Viabilities are shown relative to cells with CTRL KO followed by CTRL KO, set at 1.0. Bliss independence values were calculated using the equation EC=EA+EB−EA×EB, where EC is the expected loss of viability if the effect of 2 conditions act independently of each other, EA is the loss of viability with ERMP1 KO, and EB is the loss of viability with KDSR KO. Using this equation, synergy is defined as Eobserved>EC. For representative images, experimental replicates for FIGS. 3B-3C n=1, for d n=3, and for FIG. 3E n=3. Experimental replicates for western blot in FIG. 3F, n=1. For FIG. 3H each point represents the average of technical replicates from an independent experiment for n=3 biological replicates. In FIG. 3H, data are shown as mean+s.d. p values were calculated using two-tailed Student's t test for all statistics shown (*p<0.05, **P<0.01) #as described in Statistics, the significance between these were not determined, as in each independent set, the first bar was set as 1.0 and the other conditions are calculated relative to this.

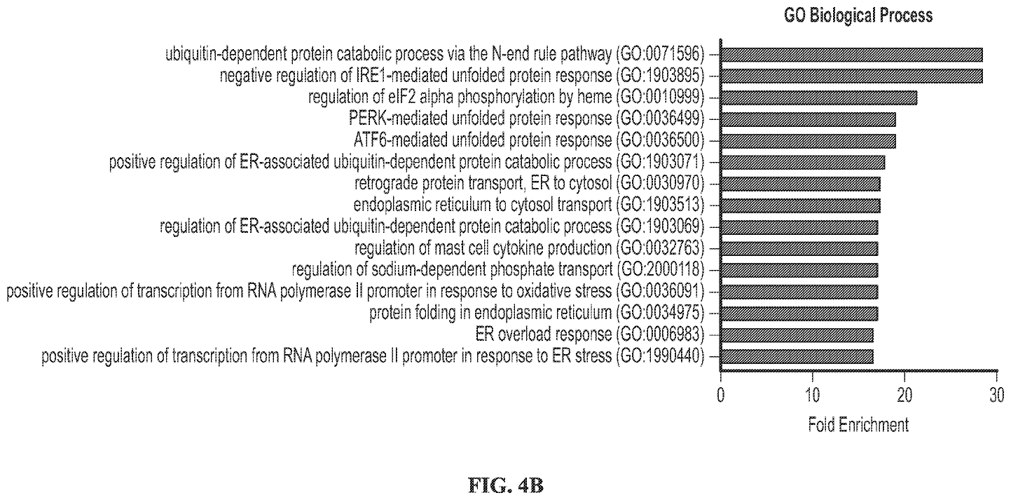

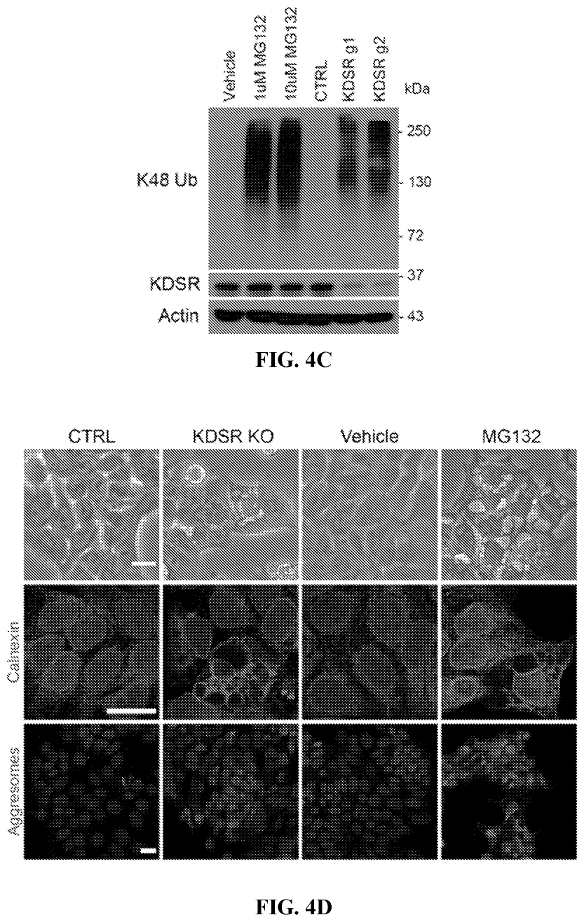

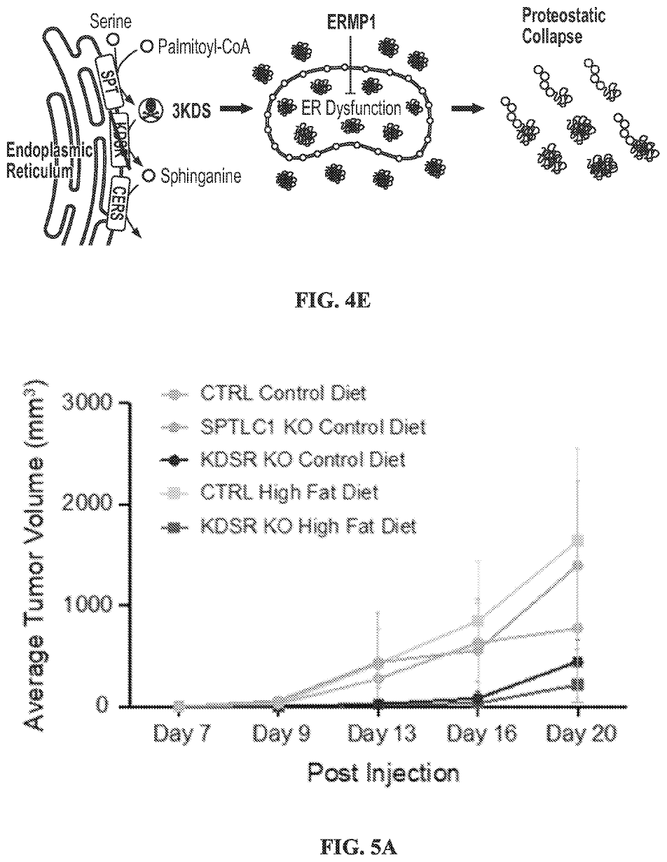

FIGS. 4A-4E: Loss of KDSR results in collapse of the ubiquitin proteasome system and misfolded protein overload. FIG. 4A: Venn diagram showing the number of common genes that were significantly upregulated at the mRNA level by RNAseq analysis in DLD1, NCIH838 (KDSR KO sensitive) and HUH7 (KDSR KO insensitive) KDSR KO cells as compared to CTRL cells. FIG. 4B: Top down list of the 15 most enriched GO Biological Processes from the list of 761 genes that were significantly upregulated in DLD1 and NCIH838 in response to KDSR KO. GO Enrichment Analysis was performed using the PANTHER Overrepresentation Test. FIG. 4C: Western blot showing the levels of K48 ubiquitinated proteins in DLD1 cells treated with the proteasome inhibitor MG132 or with KDSR KO with g1 and g2 compared to vehicle and CTRL conditions, respectively. Actin was used as the loading control. FIG. 4D: (Top panel) Representative light microscope images of the morphology of DLD1 cells treated with 3 μM MG132 (proteasome inhibitor) for 18 hr or subjected to KDSR KO, scale bar=20 μm. (Middle panel, bottom panel) Representative immunofluorescent images of DLD1 cells subjected to CTRL or KDSR KO and treatment with vehicle or 3 μM MG132 for 18 hr. Cells were immunostained with Calnexin to show ER structure (middle panel) and with aggresome dye to show protein aggregation (bottom panel). Nuclei were stained with DAPI for clarity of cell positioning, scale bars=20 μm. FIG. 4E: Proposed model for the toxicity of KDSR disruption in cancer cells. Loss of KDSR leads to accumulation of its substrate 3KDS in the ER membrane, which disrupts its structure and function. This ER dysfunction causes misfolded protein overload and impairment of the ubiquitin proteasome system, ultimately resulting in proteostatic collapse and cell death, (Created with Biorender). For RNAseq data in FIGS. 4A-4B, analysis was performed with n=3 independently prepared RNA extracts for each condition. Experimental replicates for western blot in FIG. 4C, n=1. For representative images, experimental replicates for FIG. 4D, n=1.

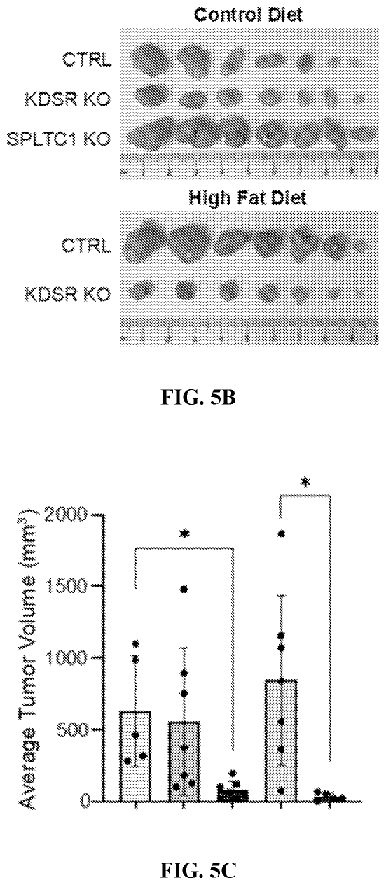

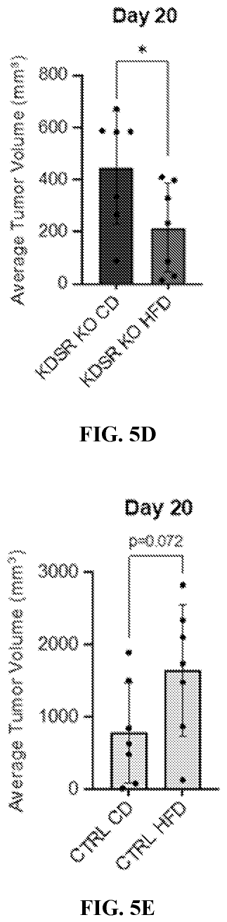

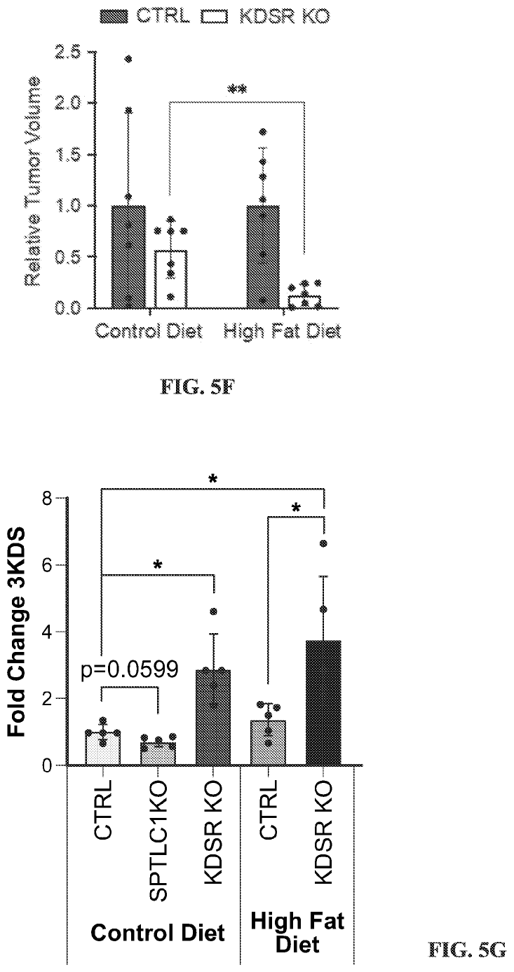

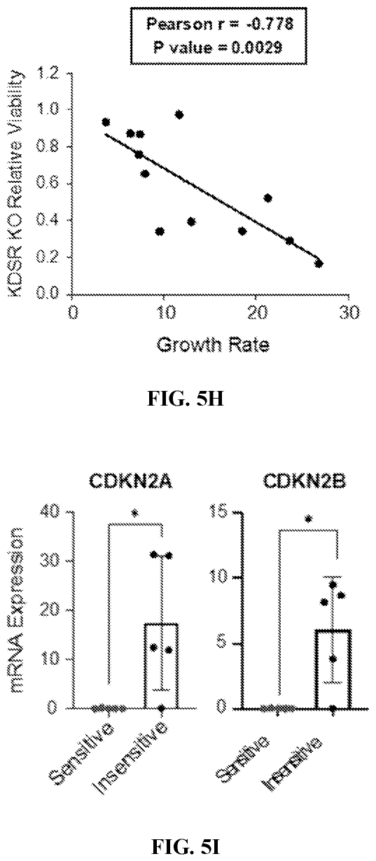

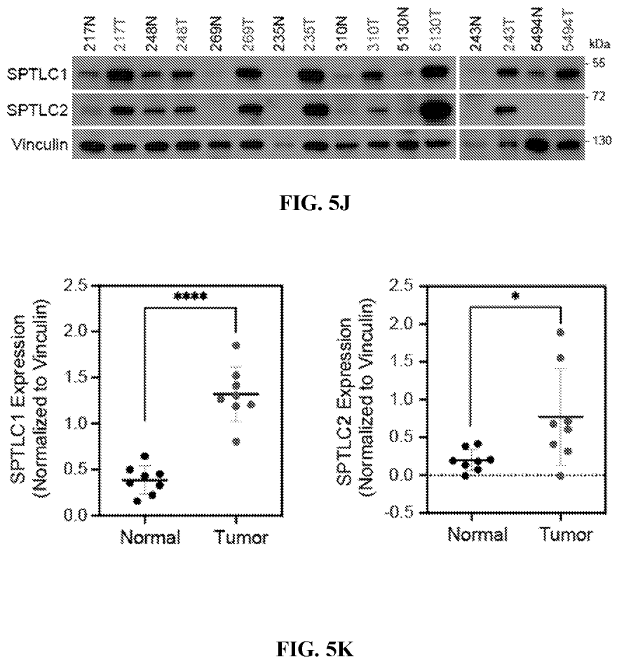

FIGS. 5A-5K: The essential detoxification function of KDSR is recapitulated in vivo and may be relevant in multiple cancer therapy contexts. FIG. 5A: Growth curve of subcutaneous xenograft model of DLD1 cells. Average volume of tumors formed over the course of 20 days after subcutaneous injection of CTRL (gray). SPTLC1 KO (gray) and KDSR KO (black) DLD1 cells into mice fed a control diet, and CTRL (gray) and KDSR KO (black) DLD1 cells into mice fed a high fat diet, n=7 mice per group. FIG. 5B: Ex vivo images of n=7 tumors in each group at the experiment endpoint. FIG. 5C: Average tumor volume of mice in each of the 5 groups at day 16 post subcutaneous injection. Tumor conditions in FIG. 5C are aligned with those shown in the graph below in FIG. 5G. FIG. 5D: Average tumor volume of KDSR KO tumors in mice fed control diet vs. those fed high fat diet at 20 days post injection. FIG. 5E: Average tumor volume of CTRL tumors in mice fed control diet vs. those fed high fat diet at 20 days post injection. FIG. 5F: Tumor volume of KDSR KO tumors (white) relative to CTRL tumors (dark gray, set at 1.0) in the control diet group and the high fat diet group to show the effect of KDSR KO under each condition. FIG. 5G: Quantification of relative 3KDS levels in tumors collected at the experiment endpoint from mice in each of the 5 groups. 3KDS levels are shown relative to CTRL control diet tumors, set at 1.0. Quantification of 3KDS was normalized to the weight of the piece of tumor that was analyzed for n=5 tumors in each group. FIG. 5H: Correlation plot of the relationship between the relative viability after KDSR KO and cell line growth rate in 12 cancer cell lines (viability data shown in FIG. 1B). Greater sensitivity to KDSR KO (lower relative viability with gene KO) significantly correlates with faster growth rate. Black line shows linear regression. Pearson r=−0.778, p=0.0029. FIG. 5I: mRNA expression of the commonly lost tumor suppressors CDKN2A and CDKN2B, in 5 KDSR KO sensitive and 5 insensitive cell lines (viability data shown in FIG. 1B). mRNA expression data for these genes is from the CCLE database from the Broad Institute. FIG. 5J: Western blot of protein levels of SPTLC1 and SPTLC2 in 8 pairs of matched normal breast tissue (black) and breast tumor tissue (gray) from patients with breast cancer. Vinculin was used as a loading control. FIG. 5K: Quantification of SPTLC1 and SPTLC2 band intensities in FIG. 5J, normalized to quantification of vinculin, to show the levels of SPTLC1 expression in normal vs. tumor tissue. Data are shown as mean±s.d. For FIG. 5A, FIG. 5C, FIGS. 5D-5G, FIG. 5I, and FIG. 5K, p values were calculated using two-tailed Student's t test (*p<0.05, **P<0.01, ****P<0.0001).

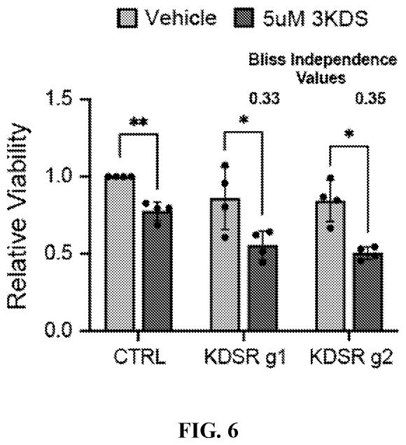

FIG. 6: Relative viability of CTRL, KDSR g1 and KDSR g2 KO DLD1 cells treated with EtOH vehicle (gray) or 5 μM 3KDS (black) for 24 hr. Synergistic effects of 3KDS and Palmitate treatment are represented by Bliss Independence values. Data are shown as mean+s.d. p values were calculated using two-tailed Student's t test for all statistics shown (*p<0.05, **P<0.01).

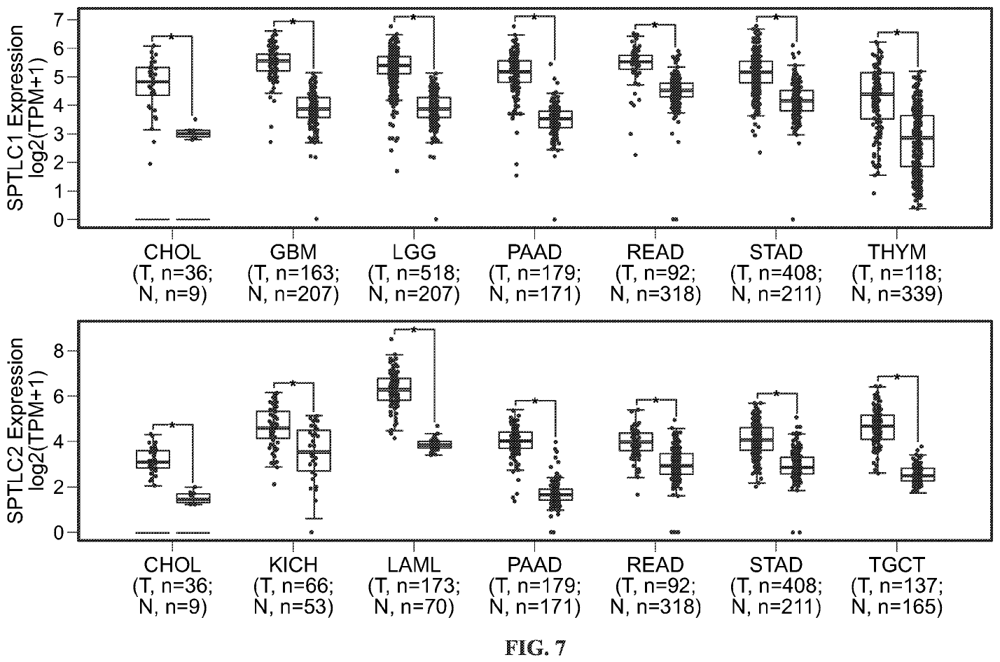

FIG. 7: Boxplots of SPLTC1 and SPTLC2 mRNA expression profiles in multiple tumor types in which expression is significantly higher in tumor (gray) compared to normal tissue (black) from TCGA and GTEx data, carried out using GEPIA2 analyses tool. CHOL=Cholangio carcinoma, GBM=Glioblastoma multiforme, LGG=Brain Lower Grade Glioma, PAAD=Pancreatic adenocarcinoma, READ=Rectum adenocarcinoma, STAD=Stomach adenocarcinoma, THYM=Thymoma, KICH=Kidney Chromophobe, LAML=Acute Myeloid Leukemia, TGCT=Testicular Germ Cell Tumors. CHOL, PAAD, READ, STAD have significantly upregulated expression of both SPTLC1 and SPTLC2.

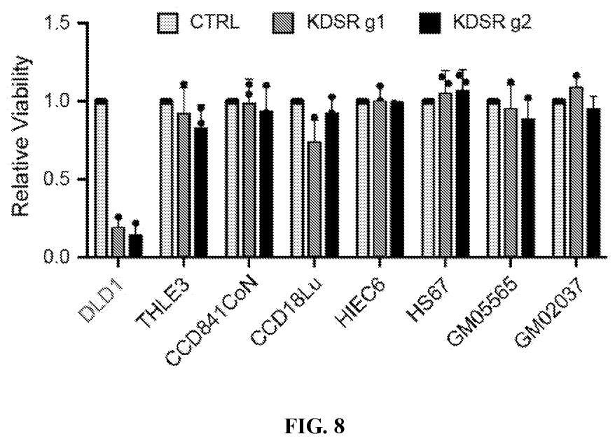

FIG. 8: Knockout of KDSR was tolerated in seven non-cancerous cell lines. Viability after 12 days of transduction with CRISPR/Cas9 targeting KDSR is shown, relative to viability after transduction with nontargeting CTRL guide. DLD1 is a colorectal adenocarcinoma cancer cell line, used as positive control for KDSR-knockout toxicity. THLE3 is a liver epithelial cell line. CCD841CON is colon epithelial cell line. CCD18Lu is a lung fibroblast cell line. HIEC6 is a small intestinal epithelial cell line. HS67 is a thymus fibroblast cell line. GM05565 and GM02037 are skin fibroblast cell lines. Light gray bars represent non-targeting control, dark grey bars represent KDSR g1, and black bars represent KDSR g2. Non-targeting control is normalized to 1. N=3 biological replicates. Mean and standard deviation are shown.

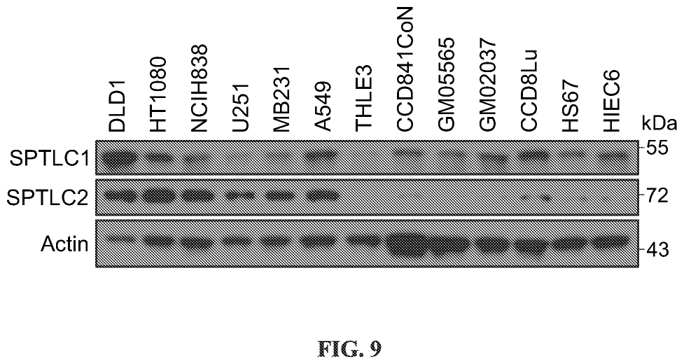

FIG. 9: Expression of SPT subunits SPTLC1 and SPTLC2 in six cancer cell lines and seven non-cancerous cell lines. Western blot of SPTLC1 and SPTLC2 protein levels are shown, with actin as a loading control.

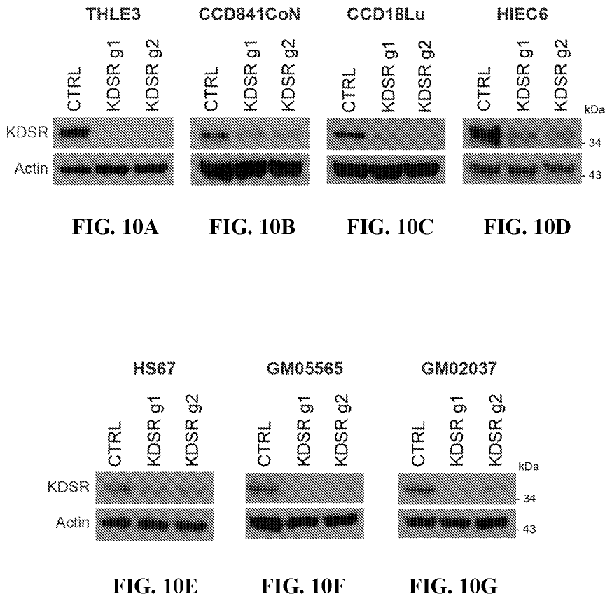

FIGS. 10A-10G: Expression of KDSR in seven non-cancerous cell lines after treatment with CRISPR/Cas9 targeting KDSR. For each of the seven cells lines, Western blot of KDSR protein levels after targeting with KDSR g1 and KDSR g2 are shown, with actin as a loading control. FIG. 10A shows KDSR protein levels for THLE3 liver epithelial cells. FIG. 10B shows KDSR protein levels for CCD841CON colon epithelial cells. FIG. 10C shows KDSR protein levels for CCD18Lu lung fibroblast cells. FIG. 10D shows KDSR protein levels for HIEC6 small intestinal epithelial cells. FIG. 10E shows KDSR protein levels for HS67 thymus fibroblast cells. FIG. 10F shows KDSR protein levels for GM05565 skin fibroblast cells. FIG. 10G shows KDSR protein levels for GM02037 skin fibroblast cells.

### DETAILED DESCRIPTION

The present disclosure is based, at least in part, on the unexpected finding that disruption of 3-ketodihydrosphingosine reductase (KDSR) induces 3-ketodihydrosphingosine (3KDS) accumulation in vivo, and dramatically impairs tumor growth, which can be synergized with a high fat diet to provide additional palmitate input for 3KDS production.

The well-established ‘signaling sphingolipids’ such as ceramides and SIP can be readily obtained via salvage, which leaves uncertain the importance of de novo biosynthesis pathway in cancer cells. In particular, little is known about the long chain base 3KDS and its processing enzyme KDSR in any biological context, let alone in cancer. Loss of function mutations in KDSR cause keratinization disorders and thrombocytopenia in patients, however the mechanisms for this are unclear⁷⁻¹⁰. The experimental data provided herein reveals that under both cell culture and xenograft tumor conditions, KDSR is not needed for producing downstream sphingolipids, but instead performs an essential detoxification function in preventing buildup of its substrate 3KDS in cancer cells. Studies described herein have shown, for the first time, a biological function for 3KDS as a toxic metabolite, which raises interesting implications for the pathology of KDSR disorders.

Furthermore, the data provided herein shows that the serine palmitoyltransferase complex (SPT: comprising SPTLC1 and SPTLC2), which produces the toxic metabolite 3KDS, is a critical determinant of dependency on KDSR and sensitivity to knockout of KDSR. As SPT expression is increased in breast cancer relative to normal tissue, and in various other cancers, this provides a basis for cancer selectivity of targeting KDSR, as shown in the Examples. SPTLC1 and SPTLC2 expression levels can also be used as biomarkers for which tumors would respond to KDSR disruption.

The experimental data provided herein also indicates a cross-talk between 3KDS production and ER function/unfolded protein response that may have biological implications in cancer cells and beyond. Maintaining correct levels of long chain bases such as sphinganine is critical for maintaining normal nuclear morphology, and deregulation of this process may be a pathological factor in trisomies such as Down syndrome¹¹. Here, a similar scenario is possible in which de novo sphingolipid biosynthesis and control of 3KDS levels, balanced between production via SPTLC1 and clearance via KDSR, regulate ER morphology either via direct biomechanical or yet to be characterized signaling mechanisms, and that KDSR disruption is a manifestation of the deregulation of this process.

Finally, the experimental data provided herein also introduces KDSR as an attractive target for poisoning cancer cells, which can be further exploited through dietary modulation, such as palmitate supplementation or ketogenic diet. The ability to increase toxic 3KDS accumulation with fatty acid supplementation, in addition to the inability to target cells through SPTLC1 due to sphingolipid salvage, highlight the critical role of the tumor microenvironment and its influence on the efficacy of a therapeutic target in cancer. Furthermore, the effect of palmitate availability on 3KDS accumulation suggests intriguing scenarios in which cancer cells with increased fatty acid/palmitate uptake, such as metastatic subtypes¹²⁻¹⁴, may be particularly vulnerable to KDSR targeting. Studies described herein demonstrated multiple additional scenarios for KDSR and cancer therapy, such as tight association of KDSR KO sensitivity with hyperproliferative cells and cells lacking CDKN2A or B tumor suppressor. Additionally, cancers such as breast cancer cells have increased expression SPTLC1, the 3KDS producing enzyme, suggesting that these cells will be selectively vulnerable to KDSR targeting.

Taken together, studies described herein identifying KDSR as a detoxifying enzyme that allows proper ER function and proteostasis sheds light on the interplay between sphingolipid biosynthesis and ER biology/proteostasis, and how deregulation of this process can be used for therapeutic means.

Accordingly, the present disclosure provides, in some aspects, therapeutic uses of KDSR disruption (e.g., disruption of the KDSR gene and/or expression of the KDSR protein) and 3KDS for treating a cancer.

### I. Inhibitors of KDSR, Precursors of 3KDS, and 3KDS

Provided herein are methods for treating cancer that involve administering an inhibitor of KDSR, a precursor of 3KDS (e.g., palmitate), 3KDS itself, or a combination thereof to a subject.

### (a) Inhibitors of KDSR

KDSR (also known as follicular variant translocation protein 1 (FVT1)) is a key enzyme in the de novo sphingolipid synthesis that catalyzes the reduction of KDS to dihydrosphingosine (DHS), which can then be converted to various ceramides by five different ceramide synthases. There are at least two isoforms of KDSR produced by alternative splicing. As used herein, the term “KDSR” encompasses KDSR isoforms as well as KDSR from any organism (e.g., human KDSR). Exemplary amino acid sequences of isoform 1 and isoform 2 of human KDSR are provided in UniProt Identifier Q06136-1 and Q06136-2, respectively.

The term “inhibitor of KDSR” or “KDSR inhibitor,” as used herein, refers to a molecule (e.g., a small molecule or a biological molecule) that blocks, inhibits, reduces (including significantly), or interferes with KDSR (e.g., mammalian KDSR such as human KDSR) biological activity in vitro, in situ, and/or in vivo. The term “inhibitor” implies no specific mechanism of biological action whatsoever, and expressly includes and encompasses all possible pharmacological, physiological, and biochemical interactions with KDSR whether direct or indirect, and whether interacting with KDSR, its substrate, or through another mechanism, and its consequences which can be achieved by a variety of different, and chemically divergent compositions.

For example, an inhibitor of KDSR can be a molecule that inhibits or disrupts KDSR itself (e.g., human KDSR), a biological activity of KDSR (e.g., including but not limited to its ability to mediate any aspect of cancer), or the consequences of the biological activity to any meaningful degree, e.g., at least 10%, at least 20%, at least 30%, at least 40%, at least 50%, at least 60%, at least 70%, at least 80%, at least 90%, or more.

Non-limiting examples of an inhibitor of KDSR of for use in the methods described herein include a small molecule, an agent that inhibits expression of KDSR (e.g., a nucleic acid molecule that inhibit KDSR expression such as a short interfering RNA (siRNA)), anti-KDSR antibody, or a peptide that inhibits KDSR (e.g., a peptide aptamer, a KDSR structural analog). In some embodiments, the KDSR inhibitor binds KDSR (i.e., physically interacts with KDSR), binds to a substrate of KDSR, and/or inhibits expression (i.e., transcription or translation) or processing of a latent form of KDSR into its active form. In some examples, the inhibitor of KDSR can be conjugated to a toxin that causes toxicity to KDSR-expressing cancer cells.

Any small molecule suitable for inhibiting KDSR can be used in methods described herein. The term “small molecule inhibitor of KDSR” refers to small organic compounds, inorganic compounds, or any combination thereof that inhibits or reduces KDSR biological activity (e.g., enzymatic activity). In some embodiments, a small molecule inhibitor of KDSR used in the methods described herein inhibits KDSR biological activity by at least 10% (e.g., at least 20%, at least 30%, at least 40%, at least 50%, at least 60%, at least 70%, at least 80%, at least 90%, or more). Small molecule inhibitors can be used in methods described herein in the free form, as a salt thereof, a prodrug derivative thereof, or combinations thereof.

Any agent suitable for inhibiting expression of KDSR can be used in methods described herein. In some embodiments, an agent that inhibits expression of KDSR used in the methods described herein inhibits KDSR expression by at least 10% (e.g., at least 20%, at least 30%, at least 40%, at least 50%, at least 60%, at least 70%, at least 80%, at least 90%, or more). In some examples, agents that inhibit expression of KDSR are nucleic acid molecules such as short interfering nucleic acid (siNA), short interfering RNA (siRNA), double-stranded RNA (dsRNA), micro-RNA (miRNA), and short hairpin RNA (shRNA) molecules. Such nucleic acid molecules can include non-naturally-occurring nucleobases (e.g., modified nucleobases), sugars (e.g., substituted sugar moieties), and/or covalent internucleoside linkages (e.g., modified backbones). An exemplary nucleic acid sequence of human KDSR is provided in NCBI Reference Sequence NM_002035.2. Nucleic acids for inhibiting expression of KDSR are commercially available, e.g., from Applied Biological Materials, OriGene, ThermoFisher, Santa Cruz Biotechnology, and VectorBuilder.

In some examples, the inhibitor of KDSR can be one or more molecules that disrupt the KDSR gene. Any molecule(s) known in the art can be used to disrupt the KDSR gene including gene editing molecules such as guide RNA (gRNA) and clustered regularly interspaced short palindromic repeat (CRISPR)-associated 9 (Cas9) nuclease.

In some examples, the inhibitor of KDSR to be used in methods described herein can be an anti-KDSR antibody. An anti-KDSR antibody is an antibody capable of binding to KDSR, which can inhibit KDSR biological activity and/or downstream pathway(s) mediated by KDSR signaling. In some embodiments, an anti-KDSR antibody used in the methods described herein inhibits KDSR biological activity and/or downstream pathway(s) mediated by KDSR signaling by at least 10% (e.g., at least 20%, at least 30%, at least 40%, at least 50%, at least 60%, at least 70%, at least 80%, at least 90%, or more). Antibodies that bind to KDSR are commercially available, e.g., from Abcam, Abnova, Atlas Antibodies, Invitrogen, LifeSpan Biosciences, Novus Biologicals, and Santa Cruz. Biotechnology.

An antibody is an immunoglobulin molecule capable of specific binding to a target, such as carbohydrate, polynucleotide, lipid, polynucleotide, lipid, polypeptide, through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof such as Fab, Fab′, F(ab′)₂, Fv, single chain (scFv), mutants thereof, fusion proteins comprising antibody portion, humanized antibodies, chimeric antibodies, diabodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An anti-KDSR antibody can be an antibody of any class, such as IgD, IgE, IgG, IgA, or IgM (or sub-class thereof), or the anti-KDSR antibody need not be of any particular class.

In some examples, the inhibitor of KDSR to be used in methods described herein can be a peptide inhibitor. For example, the inhibitor of KDSR can be a peptide comprising a portion of a KDSR-binding protein that specifically binds to KDSR and blocks its catalytic activity and/or its interaction with one or more KDSR binding proteins. In some embodiments, a peptide inhibitor used in the methods described herein inhibits KDSR biological activity and/or downstream pathway(s) mediated by KDSR signaling by at least 10% (e.g., at least 20%, at least 30%, at least 40%, at least 50%, at least 60%, at least 70%, at least 80%, at least 90%, or more).

Any of the polynucleotides and polypeptides described herein for inhibiting KDSR can be included in a delivery vehicle. The delivery vehicle can be of viral (e.g., viral vectors) or non-viral origin (e.g., eukaryotic cell delivery vehicles). Viral vectors are known in the art and include, but are not limited to, retroviral vectors, lentiviral vectors, adenoviral vectors, adeno-associated viral vectors, herpes simplex viral vectors, and vaccinia viral vectors. Expression of coding sequences in the delivery vehicle can be induced using endogenous or heterologous promoters and/or enhances. Expression of the coding sequence can be either constitutive or regulated.

### (b) 3KDS and Precursors Thereof

The present disclosure provides experimental data showing that disruption of KDSR cased a 200-fold accumulation of its substrate 3KDS and that this accumulation was toxic to cancer cells (see FIGS. 2A-2B). It was also shown that treatment of cancer cells with palmitate, which is a precursor in 3KDS production, synergized with the toxic effect of KDSR disruption to induce an 800-fold accumulation of 3KDS (see FIG. 2F).

Accordingly, provided herein, are methods and compositions for treating cancer using 3KDS and/or a 3KDS precursor such as palmitate. In some examples, 3KDS and/or a 3KDS precursor can be used in combination with a KDSR inhibitor.

As used herein, a “3KDS precursor” is a molecule that can be converted to 3KDS. Non-limiting examples of a 3KDS precursor include palmitate, palmitate-CoA, hexadecanal, and serine.

Various sources can be used to obtain 3KDS and/or a 3KDS precursor for use in methods and compositions described herein. For example, 3KDS and/or a 3KDS precursor can be extracted from cells, chemically synthesized, enzymatically synthesized, or combinations thereof. In some examples, when 3KDS and/or a 3KDS precursor is obtained from cells, the cells that have been engineered to increase production of 3KDS and/or a 3KDS precursor, e.g., by expression of one or more enzymes or proteins involved in sphingolipid biosynthesis. 3KDS and palmitate are commercially available, e.g., from Cayman Chemical and Sigma-Aldrich, respectively.

Any form of 3KDS or a 3KDS precursor can be used in methods and compositions described herein, e.g., free forms, salts, esters, and prodrugs. In some examples, 3KDS or a 3KDS precursor can be conjugated to a toxin that causes toxicity to cancer cells.

### (c) Pharmaceutical Compositions

Any inhibitor of KDSR, precursor of 3KDS, 3KDS itself, or combination thereof can be mixed with a pharmaceutically acceptable excipient (carrier) to form a pharmaceutical composition for use in treating a cancer (e.g., colorectal cancer, fibrosarcoma, lung cancer, brain cancer, breast cancer, prostate cancer). “Acceptable” means that the excipient must be compatible with the inhibitor of KDSR, the precursor of 3KDS, 3KDS, or the combination thereof (and preferably, capable of stabilizing the inhibitor of KDSR, the precursor of 3KDS, 3KDS, or the combination thereof) and not deleterious to the subject to be treated. Pharmaceutically acceptable excipients (carriers), including buffers, are well known in the art. See, e.g., Remington: The Science and Practice of Pharmacy 20th Ed. (2000) Lippincott Williams and Wilkins, Ed. K. E. Hoover.

Pharmaceutical compositions comprising an inhibitor of KDSR, a precursor of 3KDS, 3KDS, or a combination thereof to be used in the methods described herein can comprise pharmaceutically acceptable carriers, excipients, or stabilizers in the form of lyophilized formulations or aqueous solutions. Acceptable carriers, excipients, or stabilizers are nontoxic to recipients at the dosages and concentrations used, and may comprise buffers (e.g., phosphate, citrate, and other organic acids); antioxidants (e.g., ascorbic acid, methionine); preservatives (e.g., octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins (e.g., serum albumin, gelatin, immunoglobulins); hydrophilic polymers (e.g., polyvinylpyrrolidone); amino acids (e.g., glycine, glutamine, asparagine, histidine, arginine, lysine); monosaccharides, disaccharides, and other carbohydrates including glucose, mannose, or dextrans; chelating agents (e.g., EDTA) sugars (e.g., sucrose, mannitol, sorbitol); salt-forming counter-ions (e.g., sodium); metal complexes (e.g., Zn-protein complexes); and/or non-ionic surfactants (e.g., TWEEN™, PLURONICS™, polyethylene glycol (PEG)).

In some examples, the pharmaceutical compositions described herein comprise liposomes containing an inhibitor of KDSR, a precursor of 3KDS, 3KDS, or a combination thereof, which can be prepared by methods known in the art, e.g., reverse phase evaporation methods. In some examples, the inhibitor of KDSR, the precursor of 3KDS, 3KDS, or the combination thereof can be entrapped in microcapsules prepared by methods known in the art, e.g., coacervation or interfacial polymerization.

Injectable preparations, for example, sterile injectable aqueous or oleaginous suspensions, may be formulated according to the known art using suitable dispersing or wetting agents and suspending agents. Sterile injectable preparation may also be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

For preparing solid compositions such as tablets, the principal active ingredient (i.e., inhibitor of KDSR, precursor of 3KDS, 3KDS, or combination thereof) can be mixed with a pharmaceutical carrier, e.g., conventional tableting ingredients such as corn starch, lactose, sucrose, sorbital, talc, stearic acid, magnesium stearate, dicalcium phosphate or gums, and other pharmaceutical diluents, e.g., water, to form a solid preformulation composition containing a homogeneous mixture of an active ingredient. When referring to these preformulation compositions as homogeneous, it is meant that the active ingredient is dispersed evenly throughout the composition so that the composition can be readily subdivided into equally effective unit dosage forms such as tablets, pills, and capsules. This solid preformulation composition is then subdivided into unit dosage forms of the type described above containing from 0.1 to about 500 mg of the active ingredient of the present invention. The tablets or pills can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coating, such materials including a number of polymeric acids and mixtures of polymeric acids with such materials as shellac, cetyl alcohol, and cellulose acetate.

Suitable surface-active agents include, but are not limited to, non-ionic agents, such as polyoxyethylenesorbitans (e.g., Tween™20, 40, 60, 80 or 85) and other sorbitans (e.g., Span™20, 40, 60, 80, or 85). Compositions with a surface-active agent can comprise between 0.05% and 5% surface-active agent (e.g., between 0.1% and 2.5%). It will be appreciated that other ingredients may be added, for example mannitol or other pharmaceutically acceptable vehicles, if necessary.

Suitable emulsions can be prepared using commercially available fat emulsions, such as Intralipid™, Liposyn™, Infonutrol™, Lipofundin™, and Lipiphysan™. The active ingredient (i.e., inhibitor of KDSR, precursor of 3KDS, 3KDS, or combination thereof) can be either dissolved in a pre-mixed emulsion composition or alternatively it may be dissolved in an oil (e.g., soy bean oil, safflower oil, cottonseed oil, sesame oil, corn oil, or almond oil) and an emulsion formed upon mixing with a phospholipid (e.g., egg phospholipids, soy bean phospholipids or soybean lecithin) and water. It will be appreciated that other ingredients can be added, for example glycerol or glucose, to adjust the tonicity of the emulsion. Suitable emulsions will typically contain up to 20% oil, for example, between 5% and 20%. The fat emulsion can comprise fat droplets between 0.1 and 1.0 μm, e.g., 0.1 and 0.5 μm, and have a pH in the range of 5.5 to 8.0.

Pharmaceutical compositions for inhalation or insufflation include solutions and suspensions in pharmaceutically acceptable, aqueous or organic solvents, or mixtures thereof, and powders. The liquid or solid compositions can contain suitable pharmaceutically acceptable excipients as set out above. In some examples, the pharmaceutical compositions are administered by the oral or nasal respiratory route for local or systemic effect.

Compositions in preferably sterile pharmaceutically acceptable solvents can be nebulized by use of gases. Nebulized solutions can be breathed directly from the nebulizing device or the nebulizing device can be attached to a face mask, tent or intermittent positive pressure breathing machine. Solution, suspension, or powder compositions may be administered orally or nasally, from devices which deliver the formulation in an appropriate manner.

### II. Uses of Inhibitors of KDSR, Precursors of 3KDS, and 3KDS for Treating Cancer

To practice the method disclosed herein, an effective amount of a composition comprising an inhibitor of KDSR, a precursor of 3KDS, 3KDS, or a combination thereof (e.g., a pharmaceutical composition comprising an inhibitor of KDSR, a precursor of 3KDS, 3KDS, or a combination thereof) can be administered to a subject (e.g., a human patient) having or at risk for having a cancer via a suitable route (e.g., intravenous administration).

The term “subject” refers to a subject who needs treatment as described herein. In some embodiments, the subject is a human (e.g., a human patient) or a non-human mammal (e.g., cat, dog, horse, cow, goat, or sheep). A human subject who needs treatment can be a human patient having, suspected of having, or at risk for having a cancer, e.g., colorectal cancer, fibrosarcoma, lung cancer, brain cancer, breast cancer, or prostate cancer. A subject having a cancer can be identified by routine medical examination, e.g., medical examination (e.g., history and physical), laboratory tests (e.g., blood tests), imaging tests (e.g., CT scans), or biopsies. Such a subject can exhibit one or more symptoms associated with cancer, e.g., unexplained weight loss, fever, fatigue, cough, pain, skin changes, unusual bleeding or discharge, thickening or lumps in parts of the body, or combinations thereof. Alternatively or in addition to, such a subject can have one or more risk factors for cancer, e.g., family history, viral infections (e.g., herpes virus infections), genetic factors, occupational exposures, environmental exposures, smoking, air pollution, or combinations thereof.

“An effective amount” as used herein refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons, or virtually any other reason.

Empirical considerations such as the half-life of an agent will generally contribute to the determination of the dosage. Frequency of administration can be determined and adjusted over the course of therapy, and is generally, but not necessarily, based on treatment and/or suppression and/or amelioration and/or delay of a cancer (e.g., colorectal cancer, fibrosarcoma, lung cancer, brain cancer, breast cancer, or prostate cancer). Alternatively, sustained continuous release formulations of therapeutic agent may be appropriate. Various formulations and devices for achieving sustained release are known in the art.

In some embodiments, dosages of the inhibitor of KDSR, the precursor of 3KDS, 3KDS, or the combination thereof as described herein can be determined empirically in individuals who have been given one or more administration(s) of the inhibitor of KDSR, the precursor of 3KDS, 3KDS, or the combination thereof. For example, individuals are given incremental dosages of the inhibitor of KDSR, the precursor of 3KDS, 3KDS, or the combination thereof, and an indicator and/or a symptom of a cancer can be followed to assess efficacy of the inhibitor of KDSR, the precursor of 3KDS, 3KDS, or the combination thereof.

Generally, for administration of any inhibitor of KDSR, precursor of 3KDS, 3KDS, or combination thereof such as those described herein, an initial candidate dosage can be about 2 mg/kg. For example, a typical daily dosage can range from about any of 0.1 μg/kg to 3 μg/kg to 30 μg/kg to 300 μg/kg to 3 mg/kg to 30 mg/kg to 100 mg/kg or more, depending on factors described herein. For repeated administrations over several days or longer, depending on the condition, the treatment can be sustained until a desired suppression of symptoms occurs or until sufficient therapeutic levels are achieved to alleviate a cancer, or a symptom thereof. In such instances, a dosing regimen can comprise administration of an initial dose, followed by a weekly maintenance dose, or followed by a maintenance dose every other week.

Any suitable dosing regimen can be used in methods described herein. In some embodiments, the dosage regimen depends on the pattern of pharmacokinetic decay that the practitioner wishes to achieve. In some embodiments, dosing from one to four times per week can be used. In some embodiments, dosing from about 3 μg/kg to about 2 mg/kg (e.g., about 3 μg/kg, about 10 μg/kg, about 30 μg/kg, about 100 μg/kg, about 300 μg/kg, about 1 mg/kg, and about 2 mg/kg) can be used. In some embodiments, dosing frequency is once every week, every 2 weeks, every 3 weeks, every 4 weeks, every 5 weeks, every 6 weeks, every 7 weeks, every 8 weeks, every 9 weeks, or every 10 weeks, or once every month, every 2 months, or every 3 months, or longer. In some embodiments, dosing regimens can vary over time.

In some embodiments, the inhibitor of KDSR, the precursor of 3KDS, 3KDS, or the combination thereof is administered at a dose of about 0.001 mg to about 200 mg a day. For example, the inhibitor of KDSR, the precursor of 3KDS, 3KDS, or the combination thereof is administered at a dose of about 0.01 mg to about 100 mg a day, about 0.01 mg to about 50) mg a day about 0.01 mg to about 10 mg a day, or about 0.1 mg to about 10 mg a day.

In some embodiments, the appropriate dosage of inhibitor of KDSR, precursor of 3KDS, 3KDS, or combination thereof will depend on the specific inhibitor(s) (or pharmaceutical compositions thereof) used, the type and severity of cancer(s), previous therapy, the patient's clinical history and response to the inhibitor of KDSR, the precursor of 3KDS, 3KDS, or the combination thereof, and the discretion of the healthcare practitioner.

As used herein, the term “treating” refers to the application or administration of a composition including one or more active agents to a subject who has a cancer (e.g., colorectal cancer, fibrosarcoma, lung cancer, brain cancer, breast cancer, or prostate cancer), a symptom of a cancer, and/or a predisposition toward a cancer, with the purpose to cure, heal, alleviate, relieve, alter, remedy, ameliorate, improve, or affect the cancer, the symptom of the cancer, and/or the predisposition toward the cancer.

Alleviating a cancer includes delaying the development or progression of the disease, and/or reducing disease severity. Alleviating the disease does not necessarily require curative results.

As used herein, “delaying” the development of a cancer (e.g., colorectal cancer, fibrosarcoma, lung cancer, brain cancer, breast cancer, or prostate cancer) means to defer, hinder, slow; retard, stabilize, and/or postpone progression of the cancer. This delay can be of varying lengths of time, depending on the history of the cancer and/or individuals being treated. A method that “delays” or alleviates the development of a cancer and/or delays the onset of the cancer is a method that reduces probability of developing one or more symptoms of the cancer in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the cancer. Development of the cancer can be detectable and assessed using standard clinical techniques known in the art. However, development also refers to progression that may be undetectable. For purposes of this disclosure, development or progression refers to the biological course of the symptoms. “Development” includes occurrence, recurrence, and onset. As used herein, “onset” or “occurrence” of a cancer includes initial onset and/or recurrence.

In some embodiments, the inhibitor of KDSR is administered to a subject in an amount sufficient to reduce levels of KDSR biological activity by at least 10% (e.g., at least 20%, at least 30%, at least 40%, at least 50%, at least 60%, at least 70%, at least 80%, at least 90%, or more).

In some embodiments, the inhibitor of KDSR is administered to a subject in an amount sufficient to reduce levels of KDSR (e.g., KDSR protein and/or nucleic acids) by at least 10% (e.g., at least 20%, at least 30%, at least 40%, at least 50%, at least 60%, at least 70%, at least 80%, at least 90%, or more).

In some embodiments, the inhibitor of KDSR, the precursor of 3KDS, 3KDS, or the combination thereof is administered to a subject in an amount sufficient to increase levels of misfolded proteins by at least 10% (e.g., at least 20%, at least 30%, at least 40%, at least 50%, at least 60%, at least 70%, at least 80%, at least 90%, or more).

In some embodiments, the inhibitor of KDSR, the precursor of 3KDS, 3KDS, or the combination thereof is administered to a subject in an amount sufficient to increase levels of 3KDS by at least 10% (e.g., at least 20%, at least 30%, at least 40%, at least 50%, at least 60%, at least 70%, at least 80%, at least 90%, or more).

In some embodiments, the inhibitor of KDSR, the precursor of 3KDS, 3KDS, or the combination thereof is administered to a subject in an amount sufficient to inhibit tumor growth by at least 10% (e.g., at least 20%, at least 30%, at least 40%, at least 50%, at least 60%, at least 70%, at least 80%, at least 90%, or more).

In some embodiments, the inhibitor of KDSR, the precursor of 3KDS, 3KDS, or the combination thereof is administered to a subject in an amount sufficient to disrupt ER structure and/or function by at least 10% (e.g., at least 20%, at least 30%, at least 40%, at least 50%, at least 60%, at least 70%, at least 80%, at least 90%, or more).

The inhibitor of KDSR, the precursor of 3KDS, 3KDS, or the combination thereof can be administered using any suitable method for achieving delivery of the inhibitor of KDSR, the precursor of 3KDS, 3KDS, or the combination thereof to the subject in need thereof. The route of administration can depend on various factors such as the type of cancer to be treated and the site of the disease. In some embodiments, the inhibitor of KDSR, the precursor of 3KDS, 3KDS, or the combination thereof can be administered orally, topically, nasally, parenterally, buccally, or via an implanted reservoir. Parenteral administration includes, but is not limited to, subcutaneous, intracutaneous, intravenous, intramuscular, intraarticular, intraarterial, intrasynovial, intrasternal, intrathecal, intralesional, and intracranial injection or infusion techniques.

The particular dosage regimen, e.g., dose, timing, and repetition, used in methods described herein will depend on the particular subject and that subject's medical history.

In some embodiments, more than one inhibitor of KDSR can be administered to a subject in need thereof (e.g., a small molecule inhibitor and a peptide inhibitor are administered to the subject). The inhibitor of KDSR can be the same type or different from each other. At least one, at least two, at least three, at least four, or at least five different inhibitors of KDSR can be co-administered. In such instances, inhibitors of KDSR can have complementary activities that do not adversely affect each other. Inhibitors of KDSR can also be used in conjunction with other agents that serve to enhance and/or complement the effectiveness of the inhibitor. Inhibitors of KDSR can be administered alone or in combination with a precursor of 3KDS and/or 3KDS itself.

In some embodiments, the inhibitor of KDSR, the precursor of 3KDS, 3KDS, or the combination thereof is administered one or more times to the subject. Alternatively, or in addition to, the inhibitor of KDSR, the precursor of 3KDS, 3KDS, or the combination thereof can be administered as part of a combination therapy comprising the inhibitor of KDSR, the precursor of 3KDS, 3KDS, or the combination thereof and an additional therapy.

The term combination therapy, as used herein, embraces administration of these therapies in a sequential manner, that is wherein each therapy is administered at a different time, as well as administration of these therapies, or at least two of the therapies, in a substantially simultaneous manner.

Sequential or substantially simultaneous administration of each therapy can be affected by any appropriate route including, but not limited to, oral routes, intravenous routes, intramuscular routes, subcutaneous routes, and direct absorption through mucous membrane tissues. The therapies can be administered by the same route or by different routes. For example, a first therapy (e.g., the inhibitor of KDSR, the precursor of 3KDS, 3KDS, or the combination thereof) can be administered orally, and a second therapy (e.g., a chemotherapy) can be administered intravenously.

As used herein, the term “sequential” means, unless otherwise specified, characterized by a regular sequence or order, e.g., if a dosage regimen includes the administration of a first therapy and a second therapy, a sequential dosage regimen could include administration of the first therapy, before, simultaneously, substantially simultaneously, or after administration of the second therapy, but both therapies will be administered in a regular sequence or order. The term “separate” means, unless otherwise specified, to keep apart one from the other. The term “simultaneously” means, unless otherwise specified, happening or done at the same time, i.e., the therapies of the invention are administered at the same time. The term “substantially simultaneously” means that the therapies are administered within minutes of each other (e.g., within 10 minutes of each other) and intends to embrace joint administration as well as consecutive administration, but if the administration is consecutive it is separated in time for only a short period (e.g., the time it would take a medical practitioner to administer two agents separately). As used herein, concurrent administration and substantially simultaneous administration are used interchangeably. Sequential administration refers to temporally separated administration of the therapies described herein.

Combination therapy embraces the administration of the inhibitor of KDSR, the precursor of 3KDS, 3KDS, or the combination thereof in further combination with anti-cancer agents (e.g., chemotherapies, immunotherapies) as well as non-drug therapies (e.g., high fat diet, ketogenic diet, surgery, radiation therapy).

For example, when the non-drug therapy includes a high fat diet or a ketogenic diet, the subject consumes a high fat diet or a ketogenic diet. As used here, a “high fat diet” refers to a diet in which fat intake accounts for 40% or more of the total energy intake. In some examples, a high fat diet allows intake of fat in an amount of 80 g or more per kg of body weight each day.

As used herein, a “ketogenic diet” refers to a diet in which the carbohydrate content is less than, or equal to, about 5% of the total caloric intake of the subject each day. Thus, the ketogenic diet provides, as a function of total caloric intake each day, about 5% or less carbohydrate, about 30% to about 90% fat and about 5% to about 70% protein.

Non-limiting examples of chemotherapies include alkylating agents (e.g., metal salts such as cisplatin), plant alkaloids (e.g., taxanes, camptothecan analogs), antitumor antibiotics (e.g., anthracyclines, chromomycins), antimetabolites (e.g., folic acid antagonists such as methotrexate), topoisomerase inhibitors, and antineoplastics (e.g., ribonucleotide reductase inhibitors such as hydroxyurea).

Non-limiting examples of immunotherapies include immunomodulators (e.g., checkpoint inhibitors), cancer vaccines (e.g., therapeutic cancer vaccines, preventative cancer vaccines), adoptive cell therapies (e.g., CAR T cell therapies, tumor-infiltrating lymphocyte therapies), targeted antibodies (e.g., monoclonal antibodies, antibody-drug conjugates, bispecific antibodies), and oncolytic virus therapies (e.g., modified herpes simplex virus (HSV)).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described may be more fully understood, the following examples are set forth. The examples described in this application are offered to illustrate the methods and compositions provided herein and are not to be construed in any way as limiting their scope.

### Materials and Methods

The following materials and methods were used in the Examples set forth herein.

### Materials Used

Information for the materials used (antibodies, chemicals, DNA constructs, guide sequences, and other reagents) are provided in Table 1.

TABLE 1
Materials Used.

| Type of material | Source | Catalogue # |
|---|---|---|
| Antibodies (Western Blot) | | |
| Rabbit polyclonal Anti-SPTLC1 | Abcam | ab176706 |
| Rabbit polyclonal Anti-SPTLC2 | Abcam | ab23696 |
| Rabbit polyclonal Anti-KDSR | Sigma-Aldrich | HPA044884 |
| Mouse Monoclonal Anti-ß-actin | Sigma-Aldrich | A5541 |
| Rabbit monoclonal Anti-Vinculin | Abcam | ab129002 |
| Rabbit monoclonal Anti-PERK | Cell Signaling | 5683 |
| | Technology | |
| Rabbit monoclonal Anti-IRE1α | Cell Signaling | 3294 |
| | Technology | |
| Rabbit monoclonal Anti-Calnexin | Cell Signaling | 2679 |
| | Technology | |
| Rabbit monoclonal Anti-BiP | Cell Signaling | 3177 |
| | Technology | |
| Rabbit polyclonal Anti-Erol-Lα | Cell Signaling | 3264 |
| | Technology | |
| Rabbit monoclonal Anti-PDI | Cell Signaling | 3501 |
| | Technology | |
| Mouse monoclonal Anti-CHOP | Cell Signaling | 2895 |
| | Technology | |
| Rabbit monoclonal Anti-Ubiquitin, | EMD Millipore | 05-1307 |
| Lys48-Specific | | |
| Anti-mouse IgG, HRP-linked | Cell Signaling | 7076 |
| | Technology | |
| Mouse Anti-rabbit IgG-HRP | Santa Cruz | sc-2357 |
| | Biotechnology | |
| ICC Reagents | | |
| Rabbit polyclonal Anti-Calnexin | Proteintech | 10427-2-AP |
| Phalloidin-iFluor 488 | Abcam | ab176753 |
| Hoechst 33342 Nucleic acid stain | Invitrogen | H3570 |
| Goat Anti-Rabbit IgG H&L | Abcam | ab150078 |
| AlexaFluor555 | | |
| ER-Tracker Red | Invitrogen | E34250 |
| NBD Sphinganine | Avanti Polar Lipids | 810206 |
| SYTO Deep Red Nucleic acid stain | Invitrogen | S34900 |
| PROTEOSTAT Aggresome | Enzo Life Sciences | ENZ-51035 |
| Detection Kit | | |
| ProLong ™ Gold Antifade Mountant | Thermo Fisher | P36941 |
| with DAPI | Scientific | |
| Chemicals and kits | | |
| DMEM, high glucose, pyruvate | Gibco | 11995073 |
| Fetal Bovine Serum | Millipore Sigma | F2442 |
| Fetal Bovine Serum, charcoal | Thermo Fisher | A3382101 |
| stripped | Scientific | |
| Penicillin-Streptomycin (10,000 | Gibco | 15140122 |
| U/mL) | | |
| L-Glutamine (200 mM) | Gibco | 25030081 |
| Phosphate Buffered Saline | Boston Bioproducts | BM-220 |
| X-tremeGene 9 DNA Transfection | Millipore Sigma | 6365779001 |
| Reagent | | |
| Polybrene | Millipore Sigma | TR-1003-G |
| Puromycin Dihyrochloride | Gibco | A1113803 |
| Blasticidin | Thermo Fisher | R21001 |
| | Scientific | |
| Hygromycin B | Thermo Fisher | 10687010 |
| | Scientific | |
| CellTiter-Glo 2.0 Cell Viability | Promega | G9242 |
| Assay | | |
| Triton X-100 Lysis Buffer | Boston Bioproducts | BP-117 |
| Tris Buffered Saline-Tween (10X) | Boston Bioproducts | IBB-181 |
| Laemmli SDS-Sample Buffer (6X, | Boston Bioproducts | BP-111R |
| Reducing) | | |
| complete ™, Mini, EDTA-free | Millipore Sigma | 11836170001 |
| Protease Inhibitor Cocktail | | |
| Bio-Rad Protein Assay Dye Reagent | BioRad | 5000006 |
| Concentrate | | |
| MOPS-SDS Running Buffer | Boston Bio Products | BP-178 |
| Pierce ™ ECL Western Blotting | Thermo Fisher | 32106 |
| Substrate | Scientific | |
| SuperSignal ™ West Pico PLUS | Thermo Fisher | 34580 |
| Chemiluminescent Substrate | Scientific | |
| DMSO (Dimethyl Sulfoxide) | Corning | MT-25950CQC |
| 3-keto-Dihydrosphingosine HCl | Matreya | 1876 |
| Myriocin (ISP-1) | Santa Cruz | sc-201397 |
| | Biotechnology | |
| Palmitic Acid | Millipore Sigma | P0500 |
| BD Annexin V: FITC Apoptosis | Thermo Fisher | BDB556547 |
| Detection Kit I | Scientific | |
| Rneasy Mini Kit | Qiagen | 74104 |
| 4% Paraformaldehyde Solution | Thermo Fisher | AAJ19943K2 |
| | Scientific | |
| MG-132 | Millipore Sigma | 474790 |
| RIPA Buffer | Boston Bioproducts | BP-115 |
| Recombinant DNA | | |
| lentiCRISPR v2 | (Sanjana et al., 2014) | Addgene plasmid # |
| | | 52961 |
| pLV-EF1a-IRES-Blast | (Hayer et al., 2016) | Addgene plasmid # |
| | | 85133 |
| pMD154 | Scot Wolfe Lab | N/A |
| Animal Study Materials | | |
| Athymic Nude Mice | Charles River | N/A |
| | Laboratries | |
| Rodent Diet with 10% Kcal Fat | Research Diets Inc. | D12450J |
| (matching sucrose to D12492) | | |
| Rodent Diet with 60% Kcal Fat | Research Diets Inc. | D12492 |

CRISPR-Cas9 Guide RNA Sequences

| Target Gene | Guide Sequence | SEQ ID NO: |
|---|---|---|
| CTRL | ACTATCATGGCACCCAATTG | 1 |
| SPTLC1 g1 | GTGCTACTCACCTTAACCCT | 2 |
| SPTLC1 g2 | TTGGATTGTTGGATAACCCT | 3 |
| KDSR g1 | TGTGTGCAAACCAGAACAGG | 4 |
| KDSR g2 | GGCAAACTTGGATGCAGAGT | 5 |
| ERMP1 g1 | AGAGCGTCGAGAGGGAGCGG | 6 |
| ERMP1 g2 | GACGCGGAAGAGGAGCCCCG | 7 |

### Cell Lines and Cell Culture

All cell lines were cultured at 37° C. under 5% CO2 and 20% oxygen. All cell lines used were cultured in Dulbecco's Modified Eagle Medium (DMEM, high glucose, pyruvate) (Gibco #11995073) supplemented with 10% fetal bovine serum (FBS) (Sigma #F2442), 100 units/ml Penicillin-Streptomycin (Gibco #15140122), and 2 mM L-Glutamine (Gibco #25030081).

### Lentivirus Production and CRISPR-Cas9-Mediated Gene KO in Cell Lines

Guide RNA sequences for targets of interest were obtained from a study described previously to identify optimal guide sequences¹⁵ and were cloned into the lentiCRISPR v2 (pLCv2) construct¹⁶. All guide RNA sequences used are listed in Supplementary Table S1. Lentiviruses were produced by co-transfection of HEK293T cells with lentiCRISPR v2 containing guide sequences of interest, dvpr lentiviral packaging plasmid, and VSV-G envelope plasmid, with X-tremeGene 9 DNA Transfection Reagent (Roche). HEK293T cells were changed into fresh media at 18-20 hr post transfection, and the media containing lentivirus was collected from the cells at 24 hr post media change. Collected viruses were used directly for infections without concentration, and the viral titer for each virus was determined so that target cells could be infected with a<1 multiplicity of infection to avoid nonspecific toxicity of Cas9. Target cells were infected with lentiviruses and 10 μg/ml polybrene for 24 hr and were then changed into media containing puromycin (1-2 μg/ml) to select for cells that were successfully transduced and therefore obtained puromycin resistance. Cells were grown in the presence of puromycin for 4-5 days to ensure full selection.

### Cell Viability Assay

Cell viability was measured using CellTiter-Glo 2.0 Cell Viability Assay (Promega), which measures the number of viable (metabolically active) cells in culture by quantitating the amount of ATP present. CellTiter-Glo (CTG) Reagent and 96 well plates containing the cells were equilibrated to room temperature, 20 ul of CTG reagent was added to each well of cells and incubated for 20 minutes protected from light. Luminescence was read with an integration time of 1.0 sec using the Beckman Coulter DTX880 Multimode Detector plate reader.

For determination of relative viability as depicted in figures, CTG was measured at 2 different timepoints. The first viability measurement was taken as the baseline on the day after cells were plated to 96 wells, and the second viability measurement was taken at a later timepoint (1-5 days after baseline) to calculate the fold change viable cells over time for each condition (Day X CTG/Baseline CTG). These fold change values were then divided by the fold change of the control condition (=1.0) to determine the relative viability.

### Determination of the Effect of CRISPR-Cas9 Mediated Gene KO on Cell Viability

The target cell line was plated to a 6 well plate and infected with lentiviruses containing CTRL (non-targeting guide) or guides targeting the genes of interest. Cells were changed into fresh media with puromycin (1-2 μg/ml) at 24 hr post virus infection and selected for 4-5 days. At 6 days post infection, the selected cells were counted and plated to 96 well plates (each condition was plated to 3-6 technical replicate wells, depending on the format of the experiment). CTRL and KO cells were plated to 96 wells at equal concentrations (typically 400-1200 cells/well, depending on the growth rate of the cell line) to allow for consistent baseline CTG values across conditions. Baseline viability was measured by CTG at day 7 post infection (the day after cells were plated to 96 wells), and subsequently at day 12 post infection. Cell viability was calculated as the fold change CTG values over the course of 5 days of growth (day 12 CTG/Day 7 CTG). The effect of each gene KO was then determined by calculating the viability (day 12/7 ratio) of each CRISPR guide relative to that of the CTRL nontargeting guide.

### LC-MS Quantification of Sphingolipids

DLD1 cells were plated to 6 wells and infected for lentiviral transduction of lentiCRISPR v2 with nontargeting CTRL guide, KDSR g1, or SPTLC1 g1, with each condition infected in biological triplicate. Cells were selected with puromycin for 5 days and collected for analysis at 8 days post lentiviral infection. Cells were trypsinized, quickly washed 2× in cold PBS, centrifuged, and the cell pellet was collected for analysis. Lipid analysis was performed at the UCSD Lipidomics Core¹⁷. For quantification of sphingomyelin and ceramide species, samples were extracted via Butanol-Methanol (BUME) with 100 ul/sample. The lipid (top) layer was collected, solvent was removed, and samples were reconstituted in 100 ul LC solvent (18:1:1, IPA/DCM/MeOH). Samples were run on Reverse Phase-Ultra Performance Liquid Chromatography (Thermo Vanquish UPLC) on the Cortecs T3 C18 column. Mass spectrometry was performed on the Thermo Q Exactive Mass Spectrometer with MS/MS data dependent acquisition scan mode and analyzed with Lipid Data Analyzer (LDA) and LipidSearch software. All data are expressed as normalized intensities relative to exactly measured internal standards and constitute relative abundances per mg protein. For the identification of each lipid molecular species including its exact fatty acid composition, we used MS/MS footprinting techniques. Details of methods and nomenclature used were adapted from a previous study¹⁸.

Total sphingomyelin levels under each condition were calculated as the sum of 121 analytes and total ceramide levels as the sum of 81 analytes. The ratio of total sphingomyelin and ceramide levels in KDSR and SPTLC1 KO cells were calculated relative to CTRL cells as shown in FIG. 1e. The select species of sphingomyelins and ceramides shown in Extended Data FIG. 2 represent the top 10 most abundant species that were detected in all 3 biological replicate samples of the CTRL condition to show the effect of SPTLC1 and KDSR KO on the most abundant sphingolipids compared to CTRL. LC-MS quantification data of all sphingomyelin and ceramide species is shown in Table 2.

TABLE 2
LC-MS Data.

| | | MS2 | % |
|---|---|---|---|
| Class | Analytes | Confirmed | Coverage |
| CER | 121 | 18 | 15% |
| SM | 81 | 15 | 19% |
| Total | 202 | 33 | 16% |

| Sample ID | Cer | SM | Total |
|---|---|---|---|
| RATIO | | | |
| DLD1 CTRL (1) | 136.2 | 679.4 | 815.6 |
| DLD1 CTRL (2) | 145.3 | 629.2 | 774.5 |
| DLD1 CTRL (3) | 127.7 | 565.4 | 693.1 |
| DLD1 KDSR g1 (1) | 67.9 | 468.0 | 535.9 |
| DLD1 KDSR g1 (2) | 101.4 | 607.6 | 709.0 |
| DLD1 KDSR g1 (3) | 99.2 | 582.7 | 681.9 |
| DLD1 SPTLC1 g1 (1) | 66.2 | 653.9 | 720.1 |
| DLD1 SPTLC1 g1 (2) | 36.6 | 449.2 | 485.8 |
| DLD1 SPTLC1 g1 (3) | 66.9 | 453.5 | 520.4 |
| Percentage | | | |
| DLD1 CTRL (1) | 16.7% | 83.3% | 100.0% |
| DLD1 CTRL (2) | 18.8% | 81.2% | 100.0% |
| DLD1 CTRL (3) | 18.4% | 81.6% | 100.0% |
| DLD1 KDSR g1 (1) | 12.7% | 87.3% | 100.0% |
| DLD1 KDSR g1 (2) | 14.3% | 85.7% | 100.0% |
| DLD1 KDSR g1 (3) | 14.5% | 85.5% | 100.0% |
| DLD1 SPTLC1 g1 (1) | 9.2% | 90.8% | 100.0% |
| DLD1 SPTLC1 g1 (2) | 7.5% | 92.5% | 100.0% |
| DLD1 SPTLC1 g1 (3) | 12.9% | 87.1% | 100.0% |

### Lipid Free Viability Assay

DLD1 cells were grown in media with 10% FBS (normal growth condition) or 10% charcoal stripped serum (lipid free condition) for 1 week prior to lentiviral infection for CRISPR-mediated gene KO. Cells were infected in 6 wells, selected with puromycin, and plated to 96 wells as described previously in the appropriate serum conditions throughout the entirety of the experiment. Baseline CTG was measured at day 7 post infection, and subsequently at day 10 post infection. For the myriocin treated condition, CTRL cells were treated with 15 μM myriocin at day 7 post infection and were treated for 72 hr at the time of day 10 CTG measurement. Viability was calculated over the course of 3 days of growth and measured relative to CTRL untreated condition.

### Western Blot Analysis

Cells were washed in cold PBS and resuspended in 1% Triton-X 100 containing protease inhibitor cocktail and lysed on ice. Following cell lysis, samples were centrifuged at 13,000 rpm at 4° C. for 10 min to remove cell debris and the supernatant (cell lysate) was collected. The protein concentration of each lysate was measured by Bradford Assay. Western blot samples were prepared by diluting lysates in 6× Laemmli Buffer and boiled at 95° C. for 10 min. Samples were loaded to polyacrylamide gels at equal protein concentrations and analyzed by standard SDS-PAGE western blotting techniques. Protein levels were detected using HRP-conjugated secondary antibodies and chemiluminescent substrates (Pierce ECL or Pico PLUS). We found that not boiling the samples prior to gel electrophoresis was required to detect KDSR protein levels¹⁹.

### Cell Death Analysis

Cell death was measured using the Annexin V-FITC Apoptosis Detection Kit for flow cytometry (BD Biosciences). DLD1 cells were analyzed for cell death following KDSR KO at 8 and 10 days post lentiviral infection to induce CRISPR KO with CTRL nontargeting guide and 2 guides targeting KDSR. Briefly, cells were washed in PBS and stained with Annexin V-FITC and propidium iodide (PI) for 15 minutes protected from light. Cells were analyzed for the expression of Annexin V and PI immediately after staining by fluorescence associated cell sorting (FACS) using the BD LSR II flow cytometer. Each condition was infected and analyzed by flow cytometry in in biological triplicate. Flow cytometry data were analyzed using FlowJo Version 10.

### CRISPR Resistant KDSR Molecular Cloning and Rescue Experiment

A mutated version of the KDSR gene was designed to be resistant to targeting with KDSR g1 by the addition of 3 silent mutations: 1 mutation in the Protospacer Adjacent Motif (PAM) sequence, and 2 mutations in the seed sequence. This CRISPR Resistant version of the KDSR gene (CR KDSR) was synthesized by GenScript with the addition of BamHI and NotI restriction sites. CR KDSR was digested with BamHI and NotI and ligated into the expression vector pLV-EF1a-IRES-Blast. Lentiviruses were produced as described above containing blank pLV vector or pLV-CR KDSR.

To ensure the specificity of the phenotype induced by KDSR KO, we assessed the effects of KDSR KO in cells expressing blank pLV or CRISPR resistant KDSR to determine whether preventing targeting of KDSR with KDSR g1 rescues the effects of KDSR g1. We first transduced cells with lentivirus containing pLV Blank or pLV-CR KDSR and selected cells with blasticidin for 5 days. At day 6 post pLV (first) infection, we infected pLV Blank and pLV-CR KDSR cells with lentivirus containing pLCv2 CTRL or KDSR g1 and selected cells with puromycin for 5 days. At day 6 post pLCv2 (second) infection, cells were counted and plated to 96 wells at equal concentrations (400-600 cells/well). The baseline CTG measurement was taken at day 7 post pLCv2 (second) infection, and subsequent CTG timepoints were measured at days 10 and 12 post pLCv2 infection to determine cell viability over the course of 3 and 5 days of growth relative to cells expressing pLV Blank with CTRL KO.

### LC-MS Quantification of 3KDS

The levels of 3-ketodihydrosphingosine (3KDS) were quantified in both cells grown in culture and in tumor tissues from the in vivo xenograft experiment. Lipid analysis was performed at the UCSD Lipidomics Core¹⁷. For the in vitro experiment, DLD1 cells were infected for lentiviral transduction of pLCv2 with nontargeting CTRL guide, KDSR g1, or SPTLC1 g1. Cells were selected with puromycin for 5 days and were treated with the appropriate conditions (vehicle, 50 uM palmitate, 15 uM myriocin) at 6 days post infection. Cells were trypsinized, quickly washed 2× in cold PBS, centrifuged, and the cell pellet was collected for analysis at 8 days post lentiviral infection and 48 hr post treatment. 3KDS quantification was normalized to the protein content of n=3 independently prepared cell extracts for each condition.

For quantification of 3KDS in tumor samples, DLD1 xenograft tumors were collected and snap frozen at the endpoint of the in vivo experiment at 20 days post subcutaneous injection into mice. 3KDS was quantified in CTRL, KDSR KO, and SPTLC1 KO tumors in mice fed control diet and CTRL and KDSR KO tumors in mice fed high fat diet, in n=5 tumors from each group. The quantification of 3KDS was normalized to the weight of each tumor analyzed.

Samples were extracted with DCM/MeOH (1:1) and 1× internal standards were added to extracts. Samples were vortexed, sonicated for 10 minutes, and then centrifuged at 5000 rpm for 5 min. Samples were re-extracted, combined, and dried down in a speed vac. Samples were run on the ACQUITY UPLC System (Waters) on a Phenomenex Kinetex C18 column. Mass spectrometry analysis was performed on the Sciex 6500 Qtrap mass spectrometer and data were analyzed using Sciex Analyst and Multiquant software packages.

### Myriocin Rescue and Palmitate Synergy Experiments

Cells were plated to 6 wells and infected with lentiviruses containing pLCv2 CTRL or guides targeting KDSR. Cells were selected with puromycin for 4-5 days and were then counted and plated to 96 well plates at 6 days post lentiviral infection at 600-800 cells/well in technical triplicate or quadruplicate. Baseline cell viability was read at 7 days post infection, at which time the other 96 well plates (to be read at later timepoints) were treated with DMSO vehicle or 2 uM myriocin for rescue experiments or with EtOH vehicle or 50 uM palmitate for synergy experiments. For myriocin rescue experiments, subsequent CTG readings were taken at days 3 and 5 post treatment (10 and 12 days post infection) to determine the effect of SPT inhibition on KDSR KO toxicity. For synergy experiments, subsequent CTG readings were taken at 24, 48, and 72 hr post treatment (8, 9, and 10 days post infection) to measure the effect of KDSR KO and palmitate treatment on cell viability relative to CTRL vehicle treated cells.

To determine whether KDSR KO and 50 uM palmitate have a synergistic toxic effect on cells (FIG. 2C), we used the Bliss Independence Model. Bliss independence values were calculated using the equation E_{C}=E_{A}+E_{B}−E_{A}×E_{B}, where E_{C} is the expected loss of viability if the effect of 2 conditions act independently of each other, E_{A} is the loss of viability with KDSR KO, and E_{B} is the loss of viability with palmitate treatment. Using this equation, synergy is defined as E_{observed}>E_{C}, where the observed effect of the 2 conditions together is greater than the effect predicted if the 2 conditions act independently of each other.

### Double Knockout Rescue Experiment

To induce the first gene KO, cells were plated to 6 wells and infected with lentivirus containing pLCv2 CTRL or guides targeting SPTLC1 and selected with puromycin. Upon full selection, the cells were infected with pMD154 lentivirus containing CTRL guides or guides targeting KDSR to induce the second gene KO. Cells were selected with hygromycin (500 ug/ml) for 5 days and the double KO cells were plated to 96 wells at 400-600 cells/well in technical triplicate at 6 days after second infection. Baseline CTG was read at day 7 post second infection, and subsequent CTG readings for viability were measured at days 10 and 12 post second infection. The viability of double KO cells was therefore measured over the course of 3 and 5 days relative to cells targeted with CTRL nontargeting guides for both KOs.

### RNAseq Analysis

DLD1, NCIH838, and HUH7 cells were infected in 6 wells in biological triplicate with lentivirus containing pLCv2 CTRL or KDSR g1. Cells were selected with puromycin for 5 days and were changed into fresh media at 48 hr prior to the collection time point. DLD1 and HUH7 cells were collected at day 8 post lentiviral infection and NCIH838 cells were collected at day 10 post infection and RNA was isolated from the cells for n=3 for each condition (CTRL and KDSR KO for 3 cell lines). Collection timepoints for DLD1 and NCIH838 were chosen so that RNA was isolated from the cells when their morphology was visibly affected by KDSR KO, but before they start undergoing significant levels of cell death.

To identify cellular responses specific to the sensitive lines under KDSR KO, we used the Gene Ontology Resource₂₀ and PANTHER16.0 to identify GO Biological Processes that are significantly enriched in the genes that are significantly upregulated specifically in NCIH838 and DLD1. We used the list of upregulated genes to perform the PANTHER Overrepresentation Test using the Fisher's Exact Test to identify significantly enriched GO Biological Processes²¹.

### Immunocytochemistry

DLD1 cells were infected with lentivirus with pLCv2 containing CTRL or KDSR g1, selected with puromycin, and seeded to coverslips coated with poly-D-lysine (PDL) and laminin. At day 9 post lentiviral infection, cells were fixed with 4% paraformaldehyde (PFA) and fixed with 0.2% Triton-X Buffer and washed with PBS. After permeabilization, cells were stained for ER membrane with primary antibody against Calnexin at 1:500 (Proteintech, 10427-2-AP), Actin with Phalloidin-iFluor488 (Abcam, ab176753), and Nuclei with Hoechst 33342 at 5 ug/ml (Invitrogen, H3570) and washed with PBS. Cells were then stained with goat-anti rabbit AlexaFluor555 secondary antibody against Calnexin (Abcam, ab150078) and washed with PBS after staining. Coverslips were mounted to slides and imaged using the Nikon Eclipse Ti2 confocal microscope.

### NBD-Sphinganine Localization Assay

DLD1 cells were seeded to coverslips that were coated with PDL and laminin. To determine where sphinganine localizes when treated to cells, cells were treated with 1 uM NBD-Sphinganine (Avanti Polar Lipids, 810206), 1 uM ER Tracker Red (Invitrogen, E34250), and SYTO Deep Red Nucleic Acid Stain (Invitrogen, S34900) for 20 min. Cells were fixed with 4% PFA, washed with PBS, and coverslips were mounted to slides and imaged with the Nikon Eclipse Ti2 confocal microscope.

### Protein Aggresome Staining

Cells were grown on coverslips coated with PDL/laminin and were stained for protein aggresomes at 9 days post lentiviral infection for CTRL or KDSR KO or 18 hr post treatment with vehicle or 3 uM MG132. Protein aggresomes were detected using the PROTOEOSTAT Aggresome detection kit (Enzo Life Sciences), which contains a dye that fluoresces upon intercalation into the cross-beta spine of protein structures found in misfolded and aggregated proteins. For aggresome staining, cells were fixed in 4% PFA, washed in PBS, and permeabilized in 0.5% TXB on ice with gentle shaking for 30 minutes. Cells were washed in PBS following permeabilization and were subsequently incubated with the Aggresome Detection Reagent diluted 1:2000 in PBS for 45 min at room temperature protected from light. Cells were washed with PBS after staining and coverslips were mounted to microscope slides using ProLong Gold Antifade Mountant containing DAPI. Cells were imaged using the Zeiss LSM700 confocal microscope and Zen Black 2012 Software.

### Tumor Xenograft Model

Six-week-old male and female athymic nude mice were purchased from Charles River Laboratories and were housed in the animal facility at the University of Massachusetts Medical School. The growth of DLD1 (colorectal cancer) tumors were compared under CTRL, KDSR KO, and SPTLC1 KO in mice fed a control diet (Research Diets Inc., D12450J) and CTRL and KDSR KO in mice fed a high fat diet (Research Diets Inc., D1492). The mice were put onto the control and high fat diet 2 days prior to tumor injection. DLD1 cells transduced with pLCv2 CTRL, SPTLC1 g1, or KDSR g1 were resuspended in PBS and 1×10⁶ cells were injected subcutaneously into the right flank of mice at 6 days after lentiviral infection. Tumors were measured every 3-4 days using a Vernier caliper and mice were monitored regularly for appearance, activity, and body weight throughout the experiment. Mice were euthanized at 20 days after tumor injection and tumors were isolated, weighed, and snap frozen for further analysis. Tumor volume was measured using the formula 4/3π×(Length×Width×Depth)/2. Mice were randomly assigned to the 5 groups, with males and females divided among groups (4 males and 3 females or 3 males and 4 females in each group). Our experiments were done in compliance with a protocol approved by the Institutional Animal Care and Use Committee (IACUC) at University of Massachusetts Medical School.

### SPTLC1 and SPTLC2 Protein Quantification in Patient Breast Tissues

Matched breast tumor tissue and normal tissue from patients were snap frozen and stored at −80° C. upon surgical removal. These patient samples were obtained with informed consent from the University of Massachusetts Medical School Biorepository and Tissue Bank under protocol approved by the Institutional Review Board (IRB). The frozen tissues were homogenized and lysed in RIPA buffer supplemented with protease inhibitor cocktail on ice. After lysis, samples were centrifuged at 13000 rpm at 4° C. for 10 min, and supernatants were collected. Bradford assay was used to measure the protein concentration of each sample. Matched sample pairs were run side by side using standard SDS PAGE to detect SPTLC1 and 2 protein levels in normal and tumor tissue from the same patient. Vinculin was used as a loading control, and the samples were run multiple times to optimize equal detection of vinculin within each normal-tumor pair. Proteins were quantified using ImageJ by measuring band intensity and subtracting out background measurements. The band intensity of SPTLC1 and SPTLC2 were normalized to that of vinculin in the same sample to obtain a vinculin-normalized quantification of SPTLC1/2 protein levels.

### Data Preparation and Statistics

For all experiments showing relative viability, each condition was measured in at least 3 technical replicates within each experiment and each experiment was repeated at least 3 separate times (represented by individual points in bar graphs), unless otherwise indicated. FACS analysis, RNAseq analysis, and LC-MS lipid quantification were all done in biological triplicate from 3 separately infected/treated and analyzed samples. Data are presented as mean±standard deviation. Statistics were calculated using the two-tailed Student's t-test. P<0.05 is considered statistically significant, and data were marked with statistical significance as follows: *p<0.05, **P<0.01, ***p<0.001, ****P<0.0001, NS=not significant. Statistics and graphs were prepared using Microsoft Excel and Graphpad Prism. Image preparation and western blot quantification were done using FIJI Image J Version 2.0. Schematics in FIG. 1A, FIG. 2H, and FIG. 4f were created using Biorender.com.

### Example 1: Disruption of Sphingolipid Biosynthesis Enzyme KDSR is Deleterious to a Subset of Cancer Cells Despite their Capacity to Salvage Sphingolipids

The de novo sphingolipid biosynthesis pathway is initiated by the condensation of serine and palmitoyl-CoA to produce 3-ketodihydrosphingosine (3KDS) through the serine palmitoyltransferase (SPT) enzyme complex. This rate-limiting enzyme complex is made up of the critical subunit SPLTC1 and the catalytic subunit SPTLC2²²: a second catalytic subunit with more limited expression (SPTLC3) has been identified²³. 3KDS is subsequently converted to sphinganine by 3-ketodihydrosphingosine reductase (KDSR), leading to the downstream production of a complex array of sphingolipids (FIG. 1A). To determine whether de novo sphingolipid biosynthesis is required in cancer cells, the effect of CRISPR-Cas9 mediated disruption of the genes encoding the first two enzymes in the pathway, SPTLC1 and KDSR, was examined in 12 cancer cell lines of various tumor types. Disruption of SPTLC1 did not impair the viability of any of the cell lines tested, relative to a nontargeting control guide (CTRL) (FIGS. 1B-1C). In contrast, disruption of KDSR had a detrimental effect on the viability of many of the tested cell lines (FIGS. 1B-1C). Cell lines with greater than 40% reduction in viability upon KO were designated as “sensitive” and those with less than 40% reduction were designated as “insensitive” (FIG. 1D).

As SPT and KDSR are both sequential, irreplaceable enzymes in de novo sphingolipid biosynthesis, it was surprising that disruption of KDSR, but not SPTLC1, impacted cancer cell viability (FIG. 1D). It is possible that cancer cells may not require SPT because they can salvage sphingolipids from their environment. To this end, the levels of various species of ceramides and sphingomyelins, two major downstream products of sphingolipid biosynthesis, were measured in cells with SPTLC1 or KDSR KO as compared to CTRL cells. In the colorectal cancer cell line DLD1, which is insensitive to SPTLC1 KO but highly sensitive to KDSR KO, the level of total sphingomyelins did not significantly decrease under either SPTLC1 or KDSR KO (FIG. 1E). The total ceramide levels decreased under both KO conditions, but decreased to a greater extent under the non-toxic SPTLC1 KO, suggesting that this decrease is not detrimental to the cells (FIG. 1E). However, when fetal bovine serum in the media was replaced with lipid-free serum, eliminating sphingolipid salvage, SPTLC1 disruption or pharmacological inhibition with myriocin²⁴ became detrimental to cancer cell viability (FIG. 1F). These findings demonstrate that cancer cells do not require de novo sphingolipid biosynthesis through SPTLC1 because they can salvage sphingolipids from the extracellular environment. Importantly, this also indicated that the toxic effect of KDSR disruption is not due to reduced sphingolipid biosynthesis.

In contrast to the non-toxic effect of SPTLC1 disruption, loss of KDSR had distinct and severe effects on a subset of cancer cell lines, including an aberrant morphological phenotype followed by apoptotic cell death (FIGS. 1G-1H). Strikingly, it was observed that KDSR disruption induces the formation of enlarged vacuole-like subcellular structures that were seen by light microscope in KDSR-disruption sensitive, but not insensitive, cell lines prior to cell death (FIG. 1G). This suggested that KDSR plays a role in organelle integrity in cancer cells that undergo cell death in response to its loss. Overexpression of KDSR with silent mutations against KDSR guide RNA (g1) targeting rescued against both the toxicity and aberrant morphology caused by KDSR KO, validating that these effects were on-target (FIG. 1I).

Taken together, these results demonstrate that disruption of KDSR is deleterious to a subset of cancer cells despite their capacity to salvage sphingolipids from the extracellular environment.

### Example 2: KDSR is Required for 3KDS Detoxification Rather than to Prevent Sphingolipid Starvation

This required role for KDSR in cancer cell homeostasis, which contrasted the dispensable nature of SPTLC1, indicated a function of KDSR unrelated to its role in downstream sphingolipid production. Thus, KDSR may be required not for its product, but for removal of its substrate. This raised the possibility that clearance of the substrate of KDSR, 3KDS, may be important to a cancer cell. Indeed, disruption of KDSR (but not SPTLC1) caused a massive 200-fold accumulation of its substrate 3KDS (FIG. 2A and FIG. 2F). Next, cells were exogenously treated with 3KDS, which resulted in both cell toxicity and aberrant ‘vacuolar’ morphology, similar to that observed with KDSR KO (FIG. 2G). Furthermore. 3KDS treatment was even more toxic when treated to KDSR KO cells (FIG. 6), suggesting that 3KDS itself and not a downstream product of 3KDS (such as ceramides) are the culprit. Collectively, these findings suggested that 3KDS may be a toxic intermediate that requires clearance by KDSR.

The model for toxic accumulation of 3KDS described herein implies that the effects of KDSR disruption could be enhanced by increasing the production of 3KDS (FIG. 2H). Demonstrating this, treatment of cancer cells with palmitate, which upon conversion to palmitoyl-CoA can be a substrate for SPT-mediated 3KDS production, synergized with the toxic effect of KDSR KO and induced an 800-fold accumulation of 3KDS (FIG. 2C and FIG. 2F). This toxic accumulation model was further evaluated using the opposite approach, by inhibiting 3KDS production in KDSR KO cells. Both SPTLC1 KO and pharmacological inhibition with myriocin rescued against the loss of viability in cancer cells with KDSR disruption (FIGS. 2d-2E). Furthermore, SPT inhibition prevented 3KDS accumulation (FIG. 2F) and strikingly rescued the aberrant morphological phenotype in KDSR KO cells (FIG. 2G).

Collectively, these data confirm a clear toxic gain of function mechanism, where KDSR is required only when there is SPT activity creating a 3KDS detoxification demand (FIG. 2H).

### Example 3: Accumulation of 3KDS Severely Disrupts ER Structure and Function

To gain insight into how 3KDS exerts its toxic effects, cells were examined by electron microscopy to identify the aberrant structures formed in KDSR-disruption sensitive cancer cells. The structures that were initially observed by light microscope were easily discernable by electron microscope and resembled large round, disrupted ER structures (FIG. 3B). These structures were characterized as such by the appearance of their membrane-enclosed nature, ribosomes lining the structures' edges in some areas, as well as ribosomal chains in the vicinity (FIG. 3C). Immunofluorescent (IF) imaging of CTRL and KDSR KO cells showed that labeling of the ER membrane with an antibody against calnexin distinctly outlined the aberrant structures in KDSR KO cells, further indicating that 3KDS accumulation disrupts ER structure (FIG. 3D).

SPT and KDSR are located on the cytosolic surface of the ER membrane (FIG. 3A). Given that 3KDS production is ER membrane-localized and that it is a hydrophobic lipid molecule suggested that the high levels of 3KDS induced by KDSR KO may accumulate within the ER membrane, disrupting its structure. Previous studies have shown that similar sphingoid bases such as sphinganine and sphingosine are important components of organellar membranes and can affect membrane structure and permeability^{11,25,26}. As measuring the accumulation of 3KDS in the ER membrane directly is experimentally challenging, this was addressed by treating cancer cells with fluorescently labeled sphinganine, which is the sphingoid base product of KDSR, and is structurally very similar to 3KDS. It was observed that NBD-sphinganine strongly co-localized with ER tracker when treated to cells, indicating that spingoid bases can readily integrate into the ER membrane (FIG. 3E). This result, together with the localization of 3KDS production, strongly suggested a model in which 3KDS could accumulate in the ER membrane upon KDSR KO and lead to its collapse.

The accumulation of 3KDS and striking morphological changes to the ER suggested that it may be functionally compromised. Indeed, KDSR KO resulted in a robust induction of proteins involved in ER stress and the unfolded protein response (UPR), including early initiators of ER stress response pathways, PERK and IRE1α, the hallmark chaperone, BiP, and mediator of ER stress-induced cell death, CHOP (FIG. 3F).

Mining the Broad Institute's Cancer Dependency map to identify genes whose expression correlate with sensitivity to KDSR KO across hundreds of cancer cell lines (depmap.org), it was found that the gene with the strongest correlation was Endoplasmic Reticulum metallopeptidase 1 (ERMP1), a gene previously implicated in mediating the UPR and protecting against oxidative stress (FIG. 3G)²⁷. Validating the protective role of ERMP1 against KDSR KO toxicity, it was found that ERMP1 KO synergistically enhanced the toxicity of KDSR KO in cancer cells that are typically insensitive to KDSR disruption (FIG. 3H), further supporting the role of ER dysfunction in KDSR KO pathology.

### Example 4: Loss of KDSR Results in Collapse of the Ubiquitin Proteasome System and Misfolded Protein Overload

To further understanding of KDSR KO pathology described herein, RNA sequencing was performed to identify gene expression changes that occur in response to KDSR disruption. There were 762 genes that were significantly upregulated following KDSR KO in two sensitive cancer cell lines (DLD1 and NCIH838): 761 out of the 762 were not upregulated in an insensitive line (HUH7) (FIG. 4A). Analysis of the most enriched GO Biological Processes from the 761 genes revealed a clear signature of cellular responses to an overload of misfolded proteins in the ER, including ER protein folding. ER to cytosol protein transport, and induction of ubiquitin-dependent protein catabolism (FIG. 4B). This result strongly supported other data described herein indicating that the mechanism of toxicity of KDSR KO involves disruption of both the structure and function of the ER.

The ubiquitin proteasome system (UPS) is a critical protein degradation system that has been explored as an anti-cancer target due to its vital role in maintaining cellular homeostasis, and this possible link between 3KDS and the UPS was highly intriguing. Strikingly, KDSR KO induced accumulation of K48 ubiquitinated proteins in a similar manner to treatment with the proteasome inhibitor MG132, suggesting that KDSR KO disrupts proteasomal function (FIG. 4C). It was also observed that the morphological phenotype of cells treated with MG132 strongly resembled the aberrant enlarged ER structures that we identified in KDSR KO sensitive cancer cells, both by light microscope and by IF imaging of the ER membrane with calnexin (FIG. 4D-4E). Finally, both KDSR KO and MG132 treatment also induced formation of protein aggresomes (FIG. 4F), confirming both the RNAseq signature of misfolded protein accumulation as well as inhibition of the UPS by KDSR disruption.

Collectively, these data add mechanistic insight to the model for the essential role of KDSR in cancer cells provided herein: catastrophic disruption of ER structure and function resulting in misfolded protein overload and loss of proteostasis mediate the toxic effects of 3KDS accumulation in the ER membrane induced by loss of KDSR (FIG. 4G). Furthermore, this mechanism provides an unexpected link between de novo sphingolipid biosynthesis pathway and the ubiquitin proteasome system.

### Example 5: The Essential Detoxification Function of KDSR is Recapitulated In Vivo and May be Relevant in Multiple Cancer Therapy Contexts

The requirement for 3KDS detoxification by KDSR, and the lack of requirement for SPT function in cancer cells are dependent on palmitate and lipid availability, respectively, in the culture media (FIG. 1F and FIG. 2C). Therefore, it was imperative to examine the essentiality of KDSR and SPTLC1 in the metabolic environment of a tumor in vivo. In a subcutaneous xenograft model of colorectal cancer (DLD1), SPTLC1 KO did not significantly affect tumor growth, while KDSR KO significantly impaired tumor growth in mice, consistent with in vitro observations (FIG. 5a,b,c). Interestingly, feeding mice a high fat diet (HFD) to provide additional palmitate input for 3KDS production significantly enhanced the inhibitory effect of KDSR KO on tumor growth as compared to the effect in mice fed the control diet (CD), further validating our model in an in vivo context (FIGS. 5B-5C and FIG. 5F), and suggesting a means for increasing therapeutic efficacy. Although tumors in the CTRL HFD group trended toward a larger average tumor volume then those in the CTRL CD group, the KDSR KO tumors in mice fed the HFD grew to half the volume of those in mice fed the CD (FIGS. 5D-5E). It was also found that the levels of 3KDS measured in tumors collected at the endpoint of the experiment were significantly increased in KDSR KO tumors compared to CTRL in both the CD and HFD groups, with a trend toward synergistic 3KDS accumulation in HFD tumors (FIG. 5G and FIG. 7). These results validate the toxic accumulation model and demonstrate the therapeutic potential for targeting KDSR in vivo.

Further, the therapeutic implications for KDSR as a cancer-specific therapeutic target was examined. While assessing the effect of KDSR KO on the viability of a panel of 12 cancer cell lines (FIG. 1B), a significant correlative relationship was discovered, wherein cell lines with faster growth rates are more sensitive to loss of KDSR (FIG. 5H). Interestingly, it was also found that cell lines that are sensitive to KDSR disruption tend to have lower expression of the common tumor suppressors CDKN2A and CDKN2B than insensitive cells (FIG. 5I). As these tumor suppressors are important negative regulators of the cell cycle, their loss may underlie the observed correlation between sensitivity to KDSR disruption and growth rate. These findings implicate KDSR as a promising cancer-specific target, given that accelerated growth rate and/or loss of CDKN2A/B is a hallmark of many cancer cells. Lastly, it was determined that patient breast tumor tissues have significantly higher protein expression of SPTLC1 and SPTLC2, the two subunits that make up SPT, than matched normal breast tissue (FIGS. 5J-5K). This suggests that patient tumors with overexpression of SPT may produce more 3KDS, and therefore could be more susceptible to toxic 3KDS accumulation through KDSR inhibition than surrounding normal tissues. Taken together, these data point to multiple possibilities for cancer-specific therapeutic targeting of KDSR.

### Example 6: Toxicity Due to CRISPR/Cas9 KO of KDSR is Cancer-Specific and Predicted by SPT Expression

To evaluate the impact of KDSR KO by CRISPR/Cas9 on cancerous and non-cancerous cells, KDSR was targeted by two guide RNAs, KDSR g1 and KDSR g1 in seven non-cancerous cell lines: THLE3, a liver epithelial cell line: CCD841CON, a colon epithelial cell line; CCD18Lu, a lung fibroblast cell line: HIEC6, a small intestinal epithelial cell line; HS67, a thymus fibroblast cell line; GM05565, a skin fibroblast cell line; and GM02037, a skin fibroblast cell line. DLD1 is a colorectal adenocarcinoma cancer cell line, and was used as positive control for toxicity due to CRISPR/Cas9 KO of KDSR.

As shown in FIG. 8, KO of KDSR can be tolerated in a variety of non-cancerous cell lines, in contrast to the toxicity of KDSR KO in cancer cell lines including DLD1. These data indicate that toxicity of KO of KDSR, as observed for DLD1 cells, is cancer-specific. These data also indicate a possible therapeutic window for agents which knock out, inhibit, or reduce KDSR, as cancer cells depend on the presence of KDSR while non-cancerous cells do not.

In order to investigate the mechanistic basis of cancer-specific toxicity due to KO of KDSR, expression of serine palmitoyltransferase (SPT) subunits SPTCL1 and SPTCL2 protein was examined by Western blot in cancer and non-cancer cells lines. Cancer cell lines DLD1, HT 1080, NCIH838, U2% 1, MB231 and A549 had already been shown to be sensitive to KO of KDSR (see, for example, FIG. 1B). As shown in FIG. 9, the seven non-cancerous cells lines THLE3, CCD841CON, GM05565, GM02037, CCD8Lu, HS67, and HIEC6 each show substantially lower expression of SPTLC2 relative to the six cancer cell lines that are sensitive to KO of KDSR. These data suggest that in the non-cancerous cell lines, there is not substantial serine palmitoyltransferase expression, and therefore, the toxic metabolite 3KDS is not produced after KO of KDSR.

For each of the seven non-cancerous cell lines for which data are shown in FIG. 8 and FIG. 9. Western blots were performed to confirm that KDSR expression was reduced or eliminated in the cell line after CRISPR/Cas9 KO of KDSR. As shown in FIGS. 10A-10G, for each of the seven non-cancerous cell lines tested, KDSR expression was eliminated or substantially reduced after targeting of KDSR by CRISPR/Cas9 with both KDSR g1 and KDSR g2. For each experiment, actin served as a loading control.

### REFERENCES

1 Carreira, A. C. et al. Mammalian sphingoid bases: Biophysical, physiological and pathological properties. Prog Lipid Res, 100995, doi: 10.1016/j.plipres.2019.100995 (2019).
2 Gault, C. R., Obeid, L. M. & Hannun, Y. A. An overview of sphingolipid metabolism: from synthesis to breakdown. Adv Exp Med Biol 688, 1-23, doi: 10.1007/978-1-4419-6741-1_1 (2010).
3 Hannun, Y. A. & Obeid, L. M. Principles of bioactive lipid signalling: lessons from sphingolipids. Nat Rev Mol Cell Biol 9, 139-150, doi: 10.1038/nrm2329 (2008).
4 Hannun, Y. A. & Obeid, L. M. Sphingolipids and their metabolism in physiology and disease. Nat Rev Mol Cell Biol 19, 175-191, doi: 10.1038/nrm.2017.107 (2018).
5 Ogretmen, B. Sphingolipid metabolism in cancer signalling and therapy. Nat Rev Cancer 18, 33-50, doi: 10.1038/nrc.2017.96 (2018).
6 Kitatani, K., Idkowiak-Baldys, J. & Hannun, Y. A. The sphingolipid salvage pathway in ceramide metabolism and signaling. Cell Signal 20, 1010-1018, doi: 10.1016/j.cellsig.2007.12.006 (2008).
7 Bariana, T. K. et al. Sphingolipid dysregulation due to lack of functional KDSR impairs proplatelet formation causing thrombocytopenia. Haematologica 104, 1036-1045, doi: 10.3324/haematol.2018.204784 (2019).
8 Boyden, L. M. et al. Mutations in KDSR Cause Recessive Progressive Symmetric Erythrokeratoderma. Am J Hum Genet 100, 978-984, doi: 10.1016/j.ajhg.2017.05.003 (2017).
9 Liu, C., Chen, X. Y., Wu, W. Q. & Zhu, X. F. A Homozygotic Mutation in KDSR may Cause Keratinization Disorders and Thrombocytopenia: A Case Report. Chin Med Sci J 35, 278-282, doi: 10.24920/003656 (2020).
10 Takeichi, T. et al. Biallelic Mutations in KDSR Disrupt Ceramide Synthesis and Result in a Spectrum of Keratinization Disorders Associated with Thrombocytopenia. J Invest Dermatol 137, 2344-2353, doi: 10.1016/j.jid.2017.06.028 (2017).
11 Hwang, S. et al. Suppressing Aneuploidy-Associated Phenotypes Improves the Fitness of Trisomy 21 Cells. Cell Rep 29, 2473-2488 e2475, doi: 10.1016/j.celrep.2019.10.059 (2019).
12 Koundouros, N. & Poulogiannis, G. Reprogramming of fatty acid metabolism in cancer. Br J Cancer 122, 4-22, doi: 10.1038/s41416-019-0650-z (2020).
13 Nath, A., Li, I., Roberts, L. R. & Chan, C. Elevated free fatty acid uptake via CD36 promotes epithelial-mesenchymal transition in hepatocellular carcinoma. Sci Rep 5, 14752, doi: 10.1038/srep14752 (2015).
14 Pascual, G. et al. Targeting metastasis-initiating cells through the fatty acid receptor CD36. Nature 541, 41-45, doi: 10.1038/nature20791 (2017).
15 Wang, T., Wei, J. J., Sabatini, D. M. & Lander, E. S. Genetic screens in human cells using the CRISPR-Cas9 system. Science 343, 80-84, doi: 10.1126/science. 1246981 (2014).
16 Sanjana, N. E., Shalem, O. & Zhang, F. Improved vectors and genome-wide libraries for CRISPR screening. Nat Methods 11, 783-784, doi: 10.1038/nmeth.3047 (2014).
17 Quehenberger, O. et al. Lipidomics reveals a remarkable diversity of lipids in human plasma. J Lipid Res 51, 3299-3305, doi: 10.1194/jlr.M009449 (2010).
18 Hartler, J. et al. Automated Annotation of Sphingolipids Including Accurate Identification of Hydroxylation Sites Using MS (n) Data. Anal Chem 92, 14054-14062, doi: 10.1021/acs.analchem.0c03016 (2020).
19 Tsuji, Y. Transmembrane protein western blotting: Impact of sample preparation on detection of SLC11A2 (DMT1) and SLC40A1 (ferroportin). PLOS One 15, e0235563, doi: 10.1371/journal.pone.0235563 (2020).
20 Ashburner, M. et al. Gene ontology: tool for the unification of biology. The Gene Ontology Consortium. Nat Genet 25, 25-29, doi: 10.1038/75556 (2000).
21 Mi, H., Muruganujan, A., Ebert, D., Huang, X. & Thomas, P. D. PANTHER version 14: more genomes, a new PANTHER GO-slim and improvements in enrichment analysis tools. Nucleic Acids Res 47, D419-d426, doi: 10.1093/nar/gky 1038 (2019).
22 Li, S., Xie, T., Liu, P., Wang, L. & Gong, X. Structural insights into the assembly and substrate selectivity of human SPT-ORMDL3 complex. Nat Struct Mol Biol 28, 249-257, doi: 10.1038/s41594-020-00553-7 (2021).
23 Hornemann, T., Richard, S., Rutti, M. F., Wei, Y. & von Eckardstein, A. Cloning and initial characterization of a new subunit for mammalian serine-palmitoyltransferase. J Biol Chem 281, 37275-37281, doi: 10.1074/jbc.M608066200 (2006).
24 Miyake, Y., Kozutsumi, Y., Nakamura, S., Fujita, T. & Kawasaki, T. Serine palmitoyltransferase is the primary target of a sphingosine-like immunosuppressant, ISP-1/myriocin. Biochem Biophys Res Commun 211, 396-403, doi: 10.1006/bbrc. 1995.1827 (1995).
25 Contreras, F. X., Sot, J., Alonso, A. & Goñi, F. M. Sphingosine increases the permeability of model and cell membranes. Biophys J 90, 4085-4092, doi: 10.1529/biophysj. 105.076471 (2006).
26 Jiménez-Rojo, N. et al. Membrane permeabilization induced by sphingosine: effect of negatively charged lipids. Biophys J 106, 2577-2584, doi: 10.1016/j.bpj.2014.04.038 (2014).
27 Grandi, A. et al. ERMP1, a novel potential oncogene involved in UPR and oxidative stress defense, is highly expressed in human cancer. Oncotarget 7, 63596-63610, doi: 10.18632/oncotarget.11550 (2016).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a cancer, the method comprising administering to a subject in need thereof an effective amount of an inhibitor of 3-ketodihydrosphingosine reductase (KDSR), a precursor of 3-ketodihydrosphingosine (3KDS), 3KDS, or a combination thereof.

2. The method of claim 1, wherein the inhibitor of KDSR is selected from the group consisting of a small molecule inhibitor, a peptide inhibitor, an anti-KDSR antibody or antigen binding fragment thereof, and an agent that inhibits expression of KDSR.

3. The method of claim 2, wherein the agent that inhibits expression of KDSR is selected from the group consisting of short interfering nucleic acid (siNA), short interfering RNA (siRNA), double-stranded RNA (dsRNA), micro-RNA (miRNA), and short hairpin RNA (shRNA).

4. The method of claim 2, wherein the agent that inhibits expression of KDSR is a guide RNA (gRNA), and the method comprises further administering to the subject a clustered regularly interspaced short palindromic repeat (CRISPR)-associated 9 (Cas9) nuclease.

5. The method of claim 4, wherein the Cas9 nuclease is a nucleic acid encoding the Cas9 nuclease.

6. The method of claim 5, wherein the nucleic acid encoding the Cas9 nuclease is comprised in a vector.

7. The method of claim 6, wherein the vector is a viral vector.

8. The method of claim 7, wherein the viral vector is selected from the group consisting of a retroviral vector, a lentiviral vector, an adenoviral vector, an adeno-associated viral vector, a herpes simplex viral vector, and a vaccinia viral vector.

9. The method of claim 1, wherein the precursor of 3KDS is palmitate.

10. The method of claim 1, wherein the inhibitor of KDSR, the precursor of 3KDS, 3KDS, or a combination thereof is formulated in a pharmaceutical composition, which further comprises a pharmaceutically acceptable carrier.

11. The method of claim 1, wherein the cancer is selected from the group consisting of colorectal cancer, fibrosarcoma, lung cancer, brain cancer, breast cancer, and prostate cancer.

12. The method of claim 1, wherein the subject is a human patient.

13. The method of claim 1, further comprising administering to the subject an additional therapy.

14. The method of claim 13, wherein the additional therapy is selected from the group consisting of radiation therapy, surgical therapy, chemotherapy, and immunotherapy.

15. The method of claim 1, wherein the subject consumes a high fat diet or ketogenic diet.

16. The method of claim 1, further comprising determining expression levels of SPTLC1 and/or SPTLC2 in the cancer.

17. The method of claim 16, wherein expression levels of SPTLC1 and/or SPTLC2 in the cancer are predictive of the cancer's sensitivity to inhibition of KDSR.


SEQUENCE LISTING (7 sequences)

SEQ ID NO: 1 - DNA, 20 bp, synthetic construct
  source 1..20: mol_type = other DNA
actatcatggcacccaattg

SEQ ID NO: 2 - DNA, 20 bp, Homo sapiens
  source 1..20: mol_type = genomic DNA
gtgctactcaccttaaccct

SEQ ID NO: 3 - DNA, 20 bp, Homo sapiens
  source 1..20: mol_type = genomic DNA
ttggattgttggataaccct

SEQ ID NO: 4 - DNA, 20 bp, Homo sapiens
  source 1..20: mol_type = genomic DNA
tgtgtgcaaaccagaacagg

SEQ ID NO: 5 - DNA, 20 bp, Homo sapiens
  source 1..20: mol_type = genomic DNA
ggcaaacttggatgcagagt

SEQ ID NO: 6 - DNA, 20 bp, Homo sapiens
  source 1..20: mol_type = genomic DNA
agagcgtcgagagggagcgg

SEQ ID NO: 7 - DNA, 20 bp, Homo sapiens
  source 1..20: mol_type = genomic DNA
gacgcggaagaggagccccg